(19) United States
(12) Patent Application Publication
(10) Pub. No.: US 2006/0128608 A1
(43) Pub. Date: Jun. 15, 2006

(54) Novel chemical compounds and their use

(76) Inventors: Julia Cianci, Richmond (AU); Alistair Draffan, East St Kilda (AU); John N Lambert, Blackburn South (AU); Roland H Nearn, Chelsea Heights (AU); Van T. T Nguyen, Noble Park (AU)

Correspondence Address: SUGHRUE MION, PLLC, 2100 PENNSYLVANIA AVENUE, N.W., SUITE 800, WASHINGTON, DC, 20037, US

(21) Appl. No.: 10/536,504

(22) PCT Filed: Nov. 28, 2003
(86) PCT No.: PCT/AU03/01588

§ 371 Date: Dec. 19, 2005

(30) Foreign Application Priority Data
Nov. 29, 2002 (AU) 2002953095

(51) Int. Cl. A61K 38/16; A61K 31/7034; A61K 48/00; C07H 17/04; C07K 9/00; C07H 21/02

(52) U.S. Cl. 514/8; 514/36; 514/37; 514/44; 530/322; 536/16.7; 536/17.1

(57) ABSTRACT

The present invention relates to prodrugs of pharmaceutical moieties, more specifically antimicrobial agents, methods for their preparation, pharmaceutical formulations containing them and their use in the treatment of microbial infections.

The present invention relates to new chemical compounds and their use in medicine. In particular the invention concerns prodrugs of pharmaceutical moieties, more specifically antimicrobial agents, methods for their preparation, pharmaceutical formulations containing them and their use in the treatment of microbial infections.

### BACKGROUND OF THE INVENTION

The use of prodrugs as progenitors of pharmaceutical moieties is widespread and there are numerous examples of prodrug therapeutics that are converted to active drugs in vivo. Such prodrugs may be designed to improve absorption of pharmaceutical agents by, for example, the gastrointestinal tract.¹ Prodrugs may also be produced to facilitate transport across the blood-brain barrier,² provide slow release of pharmaceutically active agents , improve patient acceptance⁴ and minimise side effects.⁵

### SUMMARY OF THE INVENTION

We have now developed prodrugs of pharmaceutical moieties, more specifically antimicrobial agents. These prodrugs are designed with the specific purpose of increasing residency time at epithelial surfaces such as the lung and airways, urinary and gastrointestinal tracts, blood vessels and skin. To accomplish this, the pharmaceutical moieties are converted to prodrugs by modification with a pharmacokinetic regulator by way of a linker group that can be cleaved in vivo to expose the drug.

It is envisaged that interaction with cell membranes will also increase residence times in major organs such as the liver and central nervous system given appropriate routes of administration. Organ specific targeting groups (labile or otherwise) could also be attached to the prodrug to enhance the delivery process.

In a first aspect, the present invention provides a prodrug of the general Formula (I), (II) or (III): 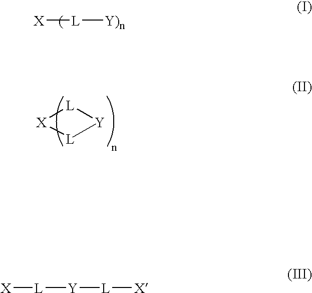
in which

X is a tobramycin moiety;

X′ is a pharmaceutically active moiety;

L is a linker group;

Y is a pharmacokinetic regulator; and

n is an integer of 1 or greater

or a pharmaceutically acceptable derivative or salt thereof.

The invention also provides a prodrug of general Formula (I), (II) or (III): 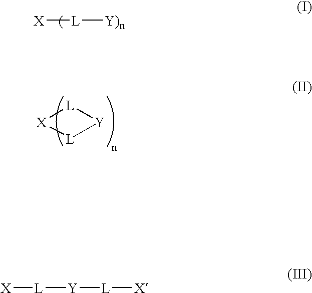
in which

X and X′ are either the same or different and selected from an aminoglycoside excluding tobramycin;

L is a linker group excluding amide and carbamate;

Y is a pharmacokinetic regulator; and

n is an integer of 1 or greater

or a pharmaceutically acceptable derivative or salt thereof.

The invention further provides a prodrug of the general Formula (I), (II) or (III): 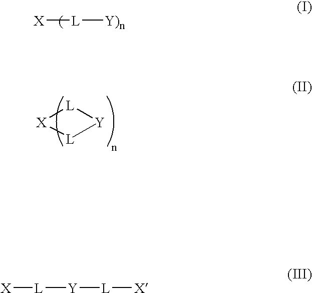
in which

X and X′ are either the same or different and selected from a nucleoside, rhinovirus capsid-binding compound, antisense oligonucleotide, peptide, an inhibitor of HIVRT, an inhibitor of influenza neuraminidase, amphotericin β, an azole and an aspartic proteinase;

L is a linker group;

Y is a pharmacokinetic regulator; and

n is an integer of 1 or greater

or a pharmaceutically acceptable derivative or salt thereof.

In a second aspect, the present invention provides a method for the preparation of the prodrug as defined above which comprises the steps of:

(a) optionally protecting the pharmaceutically active moieties X and/or X′ and/or the linker group which is attached to the optionally protected pharmacokinetic regulator Y;

(b) reacting the optionally protected pharmaceutically active moieties X and/or X′ and the optionally protected linker group L attached to the optionally protected pharmacokinetic regulator Y; and

(c) if necessary, removing the protecting groups of the pharmaceutically active moieties X and/or X′, the linker L and the pharmacokinetic regulator Y.

In a third aspect, the invention provides the prodrug as defined above or a pharmaceutically acceptable derivative thereof, for use as an active therapeutic agent in the prevention and/or treatment of a microbial infection.

A fourth aspect of the invention provides a method for the prevention and/or treatment of a microbial infection comprising the step of administering to a subject in need thereof an effective amount of the prodrug as defined above or a pharmaceutically acceptable salt or derivative thereof.

In a fifth aspect, the invention provides use of the prodrug as defined above for the manufacture of a medicament for the prevention and/or treatment of a microbial infection.

In a sixth aspect, the invention provides a method for the detection of a microbial infection which comprises the step of contacting the prodrug as defined above with a sample suspected of containing the microorganism.

In a seventh aspect, the invention provides a pharmaceutical formulation comprising the prodrug as defined above or a pharmaceutically acceptable salt or derivative thereof, together with one or more pharmaceutically acceptable carriers and, optionally, other therapeutic and/or, prophylactic ingredients.

According to an eighth aspect of the present invention there is provided an inhaler which contains the formulation as defined above.

### DETAILED DESCRIPTION OF THE INVENTION

For the purposes of this specification it will be clearly understood that the word “comprising” means “including but not limited to”, and that the word “comprises” has a corresponding meaning.

All publications, including but not limited to patents and patent applications, cited in this specification are herein incorporated by reference as if each individual publication were specifically and individually indicated to be incorporated by reference herein as though fully set forth.

The pharmaceutically-active moiety X may be selected from synthetic or natural peptides, proteins, mono- or oligosaccharides, sugar-amino acid conjugates, sugar-peptide conjugates, toxins, drugs, pro-drugs or drug like molecules. Also included for moiety X are antibodies or antigen binding fragments of whole antibody, wherein the fragments retain the binding specificity of the whole antibody molecule. The binding fragments include, for example, Fab, F(ab′)2, and Fv fragments. Binding fragments can be obtained using conventional techniques, such as proteolytic digestion of antibody by papsin or pepsin, or through standard genetic engineering techniques that are known in the art.

Indeed, the present invention is intended to encompass and be suitable for any pharmaceutically active moiety, especially any of the following drugs:

1. Analgesic anti-inflammatory agents such as, acetaminophen, aspirin, salicylic acid, methyl salicylate, choline salicylate, glycol salicylate, 1-menthol, camphor, mefanamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole and the like;

2. Drugs having an action on the central nervous system, for example sedatives, hypnotics, antianxiety agents, anticholinesterase agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivocaine, bupivacaine, etidocaine, prilocaine, benzocaine, fentanyl, nicotine, galanthamine and the like;

3. Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, cloprenaline, terfenadine, chlorpheniramine and the like;

4. Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin and the like;

5. Steroids such as, androgenic steroids, for example, testosterone, methyltestosterone, fluoxymesterone, estrogens for example, conjugated estrogens, esterified estrogens, estropipate, 17β-estradiol, 17β-estradiol esters such as 17β-estradiol valerate, equilin, mestranol, estrone, estriol, 17β-estradiol derivatives such as 17β-ethinyl estradiol, diesthylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17α-hydroxyprogesterone, dydrogesterone, dimethisterone, ethinylestrenol, norgestrel, demegestone, promegestone, megestrol acetate and the like;

6. Respiratory agents such as, theophylline and β₂-adrenergic agonists, for example, albuterol, terbutaline, metaproterenol, ritodrine, carbuterol, fenoterol, quinterenol, rimiterol, solmefamol, soterenol, tetroquinol and the like;

7. Sympathomimetics such as, dopamine, norepinephrine, penylpropanolamine, pheylephrine, psuedoephedrine, amphetamine, propylhexedrine, arecoline and the like;

8. Antimicrobial or antiinfective agents including antibacterial agents, antifungal agents, antiparasitic agents, antimycotic agents and antiviral agents, such as, those listed in the Ashgate Handbook of Anti-Infective Agents (Ed G.W.A. Milne, Ashgate Publishing, 2000), for example, tetracyclines such as oxytetracycline; penicillins such as ampicillin; cephalosporins such as cefalotin; aminoglycosides such as kanamycin A to C, amikacin, neomycin, tobramycin, streptomycin, neamine, paromomycin, lividomycin, 2230-C, ribostamycin, xyllostasin, butirosin, 4′-deoxybutyrosin, LL-BM408a, gentamycins and nebramycin; macrolides such as erythromycin, chloramphenicol, iodides, nitrofrantoin; antifungals such as clotrimazole, miconazole, chloramphenicol, nystatin, amphotericin, fradiomycin, sulfonamides, purrolnitrin, sulfacetamide, sulfamethazine, sulfadiazine, sulfamerazine, sulfamethizole and sulfisoxazole; antivirals such as inhibitors of influenza neuraminidase and idoxuridin; clarithromycin; and other anti-infectives including nitrofurazone and the like;

9. Antihypertensive agents such as, clonidine, α-methyldopa, reserpine, syrosingopine, rescinnamine, cinnarizine, hydrazine, prazosin and the like;

10. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone and the like;

11. Cardiotonics such as, digitalis, ubidecarenone, dopamine and the like;

12. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine and the like;

13. Vasoconstrictors such as, dihydroergotamine, dihydroergotoxine and the like;

14. β-blockers or antiarrhythmic agents such as, timolol pindolol, propranolol and the like;

15. Calcium antagonists and other circulatory organ agents, such as, aptopril, diltiazem, nifedipine, nicardipine, verapamil, bencyclane, ifenprodil tartarate, molsidomine, clonidine, prazosin and the like;

16. Anti-convulsants such as, nitrazepam, meprobamate, phenytoin and the like;

17. Agents for dizziness such as, isoprenaline, betahistine, scopolamine and the like;

18. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam and the like;

19. Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranquilizers such as, chlorprathixene, thiothixene, haloperidol, bromperidol, loxapine, and molindone, as well as, those agents used at lower doses in the treatment of nausea, vomiting and the like;

20. Muscle relaxants such as, tolperisone, baclofen, dantrolene sodium, cyclobenzaprine and the like;

21. Drugs for Parkinson's disease, spasticity, and acute muscle spasms such as levodopa, carbidopa, amantadine, apomorphine, bromocriptin, selegiline (deprenyl), trihexyphenidyl hydrochloride, benztropine mesylate, procyclidine hydrochloride, baclofen, diazepam, dantrolene and the like;

22. Respiratory agents such as, codeine, ephedrine, isoproterenol, dextromethorphan, orciprenaline, ipratropium bromide, cromglycic acid and the like;

23. Non-steroidal hormones or antihormones such as, corticotropin, oxytocin, vasopressin, salivary hormone, thyroid hormone, adrenal hormone, kallikrein, insulin, oxendolone and the like;

24. Vitamins such as, vitamins A, B, C, D, E and K and derivatives thereof, calciferols, mecobalamin, and the like for dermatological use;

25. Antitumor agents such as, 5-fluorouracil and derivatives thereof, krestin, picibanil, ancitabine, cytarabine and the like;

26. Enzymes such as, lysozyme, urokinaze and the like;

27. Herb medicines or crude extracts such as, glycyrrhiza, aloe, Sikon (Lithospermi radix) and the like;

28. Miotics such as pilocarpine and the like;

29. Cholinergic agonists such as, choline, acetylcholine, methacholine, carbachol, bethanechol, pilocarpine, muscarine, arecoline and the like;

30. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eycatropine and the like;

31. Mydriatics such as, atropine, cyclopentolate, homatropine, scopolamine, tropicamide, eucatropine, hydroxyamphetamine and the like;

32. Psychic energizers such as, 3-(2-aminopropyl)indole, 3-(2-aminobutyl)indole and the like;

33. Humoral agents such as, the prostaglandins, natural and synthetic, for example, PGE₁, PGE_{2α}, and PGF_{2α}, and the PGE₁ analog misoprostol.

34. Antispasmodics such as, atropine, methantheline, papaverine, cinnamedrine, methscopolamine and the like;

35. Antidepressant drugs such as, isocarboxazid, phenelzine, tranylcypromine, imipramine, amitriptyline, trimipramine, doxepin, desipramine, nortriptyline, proptriptyline, amoxapine, maprotiline, trazodone and the like;

36. Anti-diabetics such as, insulin, and anticancer drugs such as, tamoxifen, methotrexate and the like;

37. Anorectic drugs such as, dextroamphetamine, methamphetamine, phenylpropanolamin, fenfluramine, diethylpropion, mazindol, phentermine and the like;

38. Anti-allergenics such as, antazoline, methapyrilene, chlorpheniramine, pyrilamine, pheniramine and the like;

39. Decongestants such as, phenylephrine, ephedrine, naphazoline, tetrahydrozoline and the like;

40. Antipyretics such as, aspirin, salicylamide and the like;

41. Antimigrane agents such as, dihydroergotamine, pizotyline and the like;

42. Anti-malarials such as, the 4-aminoquinolines, alphaaminoquinolines, chloroquine, pyrimethamine and the like;

43. Anti-ulcer agents such as, misoprostol, omeprazole, enprostil, allantoin, aldioxa, alcloxa, N-methylscopolamine methylsulfate and the like;

44. Peptides such as, growth releasing factor and the like;

45. Anti-estrogen or anti-hormone agents such as, tamoxifen or human chorionic gonadotropin and the like.

Preferably, the pharmaceutically active moiety is an antimicrobial agent, more preferably an antibacterial agent such as an aminoglycoside, a beta-lactam antibiotic, vancomycin or ciprofloxacin. Examples of aminoglycosides include the following: 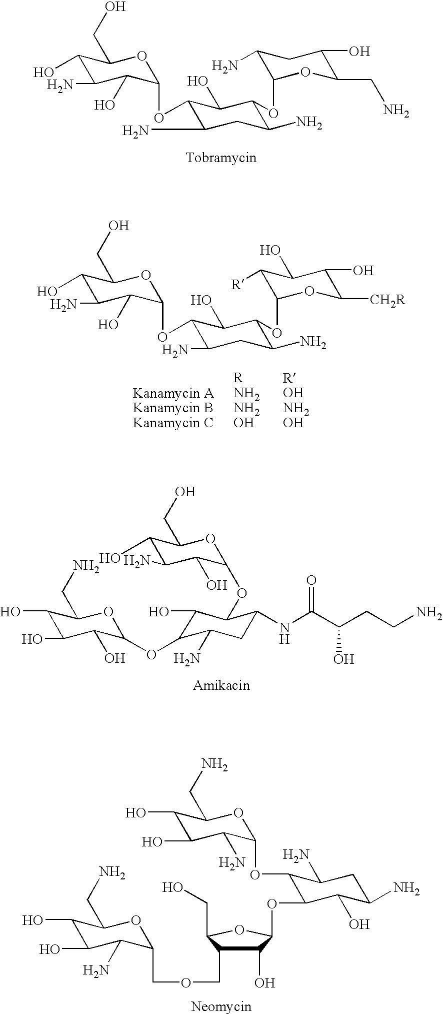
streptomycin, neamine, paromomycin, lividomycin, 2230-C, ribostamycin, xyllostasin, butirosin, 4′-deoxybutyrosin, LL-BM408a, gentamycin A, B? and nebramycin. Preferably the aminoglycoside is tobramycin, kanamycin A to C, amikacin and neomycin, more preferably tobramycin.

The antimicrobial agent may also be selected from antiviral agents, for example, nucleosides, rhinovirus capsid-binding compounds, antisense oligonucleotides, peptides, inhibitors of HIVRT and inhibitors of influenza neuraminidase, for example, a compound of Formula (A)

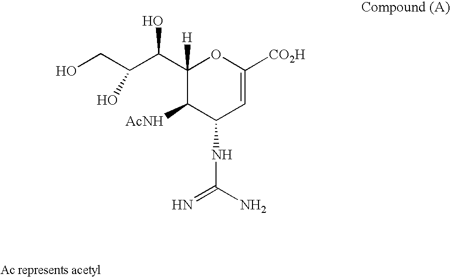

Ac represents acetyl; antifungal agents such as amphotericin β or azoles, for example, fluconazole or ketaconazole; or antiparasitic agents such as aspartic proteinases.

The term “linker group” is used herein in its broadest sense and refers to a functional group capable of being cleaved in vivo to expose the pharmaceutical moiety X. Suitable linker groups include esters, amides, ureas, thioureas, imines, acetals, ethers, phosphates, phosphate esters or diesters, thioesters, oximes and hydrazones. Preferably the linker group is an ester, amide, oxime or phosphate ester, more preferably an ester.

It will be appreciated that functional groups on the pharmaceutically active moiety form part of the linker group. Suitable points of attachment include one or more positions on the pharmaceutically active moiety containing leaving groups such as amines, hydroxyls, thiols, carboxyls, aldehydes and ketones, more preferably hydroxyls and amines. When the pharmaceutically active moiety is the aminoglycoside tobramycin, then the linker may be attached at one or more of positions 6″, 4″, 2″, 5 and 4′ which contain hydroxyl functional groups or one or more of positions 3″, 1, 3, 2′ and 6′ which contain amine groups. The 6″-OH and the 6′-NH₂ are the preferred points of attachment on tobramycin as they are the most accessible positions on this moiety from a synthetic standpoint.

The term “pharmacokinetic regulator” is used herein in its broadest sense and refers to a moiety which is capable of regulating residency time and the intensity of release of the pharmaceutical moiety X. The pharmacokinetic regulator may be a hydrophobic or hydrophilic moiety, preferably a hydrophobic moiety.

Suitable hydrophobic moieties include optionally substituted straight chain, branched and cyclic saturated or unsaturated hydrocarbons such as optionally substituted alkyl or optionally substituted alkenyl having 1 to 24 carbon atoms, preferably 1 to 20 carbon atoms, more preferably 1 to 16 carbon atoms, which are optionally interrupted with oxygen or nitrogen; optionally substituted aryl; or optionally substituted heterocyclyl.

The term “C₁₋₂₄ alkyl” refers to straight chain, branched chain or cyclic hydrocarbon groups having 1 to 24 carbon atoms. Illustrative examples of straight chain or branched chain alkyl include ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-. 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include cyclopropyl, cyclobutyl, cyclopentyl, cyclohexyl, cycloheptyl, cyclooctyl, cyclononyl and cyclodecyl and the like.

The term “C₂₋₂₄ alkenyl” refers to straight chain, branched chain or cyclic hydrocarbon groups having from 2 to 24 carbon atoms and having in addition one or more double bonds, of either E or Z stereochemistry where applicable. Examples of straight chain and branched chain alkenyl include vinyl, allyl, 1-methylvinyl, butenyl, iso-butenyl, 3-methyl-2-butenyl, 1-pentenyl, 1-hexenyl, 3-hexenyl, 1-heptenyl, 3-heptenyl, 1-octenyl, 1-nonenyl, 2-nonenyl, 3-nonenyl, 1-decenyl, 3-decenyl, 1,3-butadienyl, 1,4-pentadienyl, 1,3-hexadienyl, 1,4-hexadienyl, geranyl, farnesyl and the like. Examples of cyclic alkenyl include cyclopentenyl, 1-methyl-cyclopentenyl, cyclohexenyl, cycloheptenyl, cyclooctenyl, 1,3-cyclohexadienyl, 1,4-cyclohexadienyl, 1,3-cycloheptadienyl 1,3,5-cychloheptatrienyl, 1,3,5,7-cycloocta-tetraenyl and the like.

The term “aryl” refers to single, polynuclear, conjugated and fused residues of aromatic hydrocarbons. Examples include phenyl, biphenyl, terphenyl, quaterphenyl, phenoxyphenyl, naphthyl, tetrahydronaphthyl, indane anthracenyl, dihydroanthracenyl, benzantracenyl, dibenzanthracenyl, phenanthrenyl and the like.

The term “heterocyclyl” refers to mono- or poly-cyclic saturated, partially unsaturated and unsaturated hydrocarbon radicals containing at least one heteroatom selected from nitrogen, sulphur and oxygen. Suitable heterocyclyl groups include N-containing heterocyclic groups, such as, unsaturated 3 to 6 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl;

saturated 3 to 6-membered heteromonocyclic groups containing 1 to 4 nitrogen atoms, such as, pyrrolidinyl, imidazolidinyl, piperidino or piperazinyl;

unsaturated condensed heterocyclic groups containing 1 to 5 nitrogen atoms, such as, indolyl, isoindolyl, indolizinyl, benzimidazolyl, quinolyl, isoquinolyl, indazolyl, benzotriazolyl or tetrazolopyridazinyl;

unsaturated 3 to 6-membered heteromonocyclic group containing an oxygen atom, such as, pyranyl or furyl;

unsaturated 3 to 6-membered heteromonocyclic group containing 1 to 2 sulphur atoms, such as, thienyl;

unsaturated 3 to 6-membered heteromonocyclic group containing 1 to 2 oxygen atoms and 1 to 3 nitrogen atoms, such as, oxazolyl, isoxazolyl or oxadiazolyl;

saturated 3 to 6-membered heteromonocyclic group containing 1 to 2 oxygen atoms and 1 to 3 nitrogen atoms, such as, morpholinyl;

unsaturated condensed heterocyclic group containing 1 to 2 oxygen atoms and 1 to 3 nitrogen atoms, such as, benzoxazolyl or benzoxadiazolyl;

unsaturated 3 to 6-membered heteromonocyclic group containing 1 to 2 sulphur atoms and 1 to 3 nitrogen atoms, such as, thiazolyl or thiadiazolyl;

saturated 3 to 6-membered heteromonocyclic group containing 1 to 2 sulphur atoms and 1 to 3 nitrogen atoms, such as, thiazolidinyl; and

unsaturated condensed heterocyclic group containing 1 to 2 sulphur atoms and 1 to 3 nitrogen atoms, such as, benzothiazolyl or benzothiadiazolyl.

In this specification “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, alkynyloxy, aryloxy, carboxy, benzyloxy haloalkyl, haloalkenyloxy, haloalkynyloxy, haloaryloxy, nitro, nitroalkyl, nitroalkenyl, nitroalkynyl, nitroaryl, nitroheterocyclic, azido, amino, alkylamino, alkenylamino, alkynylamino, arylamino, benzylamino, acyl, alkenylacyl, alkynylacyl, arylacyl, acylamino, acyloxy, aldehydo, alkylsulphonyl, arylsulphonyl, alkylsulphonylamino, arylsulphonylamino, alkylsulphonyloxy, arylsulphonyloxy, heterocyclyl, heterocycloxy, heterocyclylamino, haloheterocyclyl, alkylsulphenyl, arylsulphenyl, carboalkoxy, carboaryloxy, mercapto, alkylthio, arylthio, acylthio and the like. Preferably the optional substituents are alkyl, alkenyl, alkynyl, aryl, aryloxy, arylacyl, acylamino, acyloxy, nitro, hydroxy, halo and heterocyclyl.

Examples of hydrophilic moieties include oligonucleotides up to 20 nucleotides in length, peptides up to 20 amino acids in length, peptide mimics, carbohydrates, oligosaccharides and derivatives thereof.

Preferably the pharmacokinetic regulator is a hydrophobic moiety which is selected from C₁₋₂₀ optionally substituted alkyl; C₂₋₂₀ optionally substituted alkenyl; optionally substituted aryl; or optionally substituted heterocyclyl. The alkyl or alkenyl groups may be interrupted with O, C═O, NH, optionally substituted aryl or optionally substituted heterocyclyl and may be optionally substituted with carboxyl, optionally substituted C₁₋₆ alkyl, amino or hydroxyl. The optionally substituted aryl is preferably an optionally substituted 6-membered aryl such as optionally substituted phenyl or optionally substituted biphenyl and the heterocyclyl is preferably a 5- or 6-membered heterocyclic group such as pyridyl, indolyl, indazolyl, 2,3-dihydro-1H-indolyl, furanyl, isoxazolyl, pyrazolyl and thiofuranyl. The optional substituents on the phenyl or heterocyclyl are preferably halo, C₁₋₄ alkyl, C₁₋₄ alkoxy, hydroxy and OCF₃.

It will be appreciated by those skilled in the art that the prodrugs of Formula (I), (II) or (III) may be modified to provide pharmaceutically acceptable derivatives thereof at any one or more of the functional groups in the prodrugs of Formula (I), (II) or (III). Of particular interest as such derivatives are prodrugs modified at the carboxyl function, hydroxyl function or at amino groups. Thus, prodrugs of interest include alkyl esters, such as methyl, ethyl, propyl or isopropyl esters, aryl esters, such as phenyl, benzoyl esters, and acetyl esters of the prodrugs of Formula (I), (II) or (III).

The term “pharmaceutically acceptable derivative” means any pharmaceutically acceptable salt, hydrate, ether, ester, amide, active metabolite, analogue, residue or any other compound which is not biologically or otherwise undesirable and induces the desired pharmacological and/or physiological effect.

Pharmaceutically acceptable salts of the prodrugs of Formula (I), (II) or (III) include those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids. Other acids such as oxalic acid, while not in themselves pharmaceutically acceptable, may be useful in the preparation of salts useful as intermediates in obtaining prodrugs of the invention and their pharmaceutically acceptable acid addition salts.

Salts derived from appropriate bases include alkali metal (eg. sodium), alkaline earth metal (eg. magnesium), ammonium, and NR₄⁺ (where R is C₁₋₄ alkyl) salts.

The prodrugs of the invention may be prepared by methods described herein. It will be apparent to those skilled in the art, that it may be necessary to use protecting groups to protect one or more functional groups of the pharmaceutically active moiety during the process of attaching the pharmaceutical moiety to the linker group and the pharmacokinetic regulator. See for example “Protective Groups in Organic Synthesis” by T. W. Green and P.G.M. Nuts (John Wiley & Sons, 1991).

The chemistry of the linking reaction will be determined either by the nature of reactive functional groups present in the pharmaceutical moiety or the nature of reactive groups that can be introduced to the pharmaceutical moiety using a series of chemical transformations. General methods for preparing the prodrugs will now be described with reference to the nature of the functional group present in or introduced to the pharmaceutical moiety. It should be noted that many of the prodrugs described herein can be prepared using either conventional chemical methods or enzymatic methods. Enzymatic methods can in some instances provide greater selectivity than conventional methods. It will be appreciated that the invention is not limited to such methods.

Pharmaceutical Moieties Bearing Amines or to Which Amines can be Readily Introduced

1) Amide formation
a) Conventional chemical means
b) Enzymatic peptide coupling using, for example, thermolysin

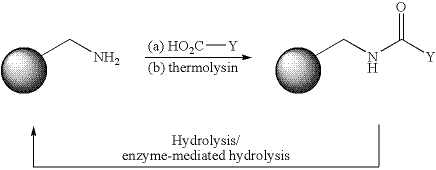

2) Urea or thiourea formation

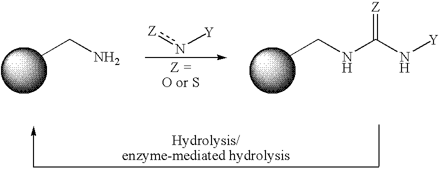

3) Imine formation 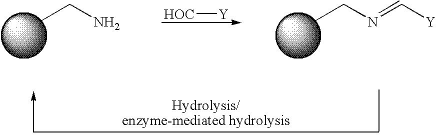
Pharmaceutical Moieties Bearing Alcohols or to Which Alcohols can be Readily Introduced

1) Ester Formation
a) Conventional chemical means 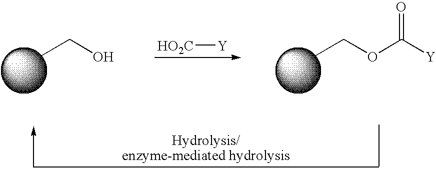
b) Transesterification using, for example, lipase enzymes

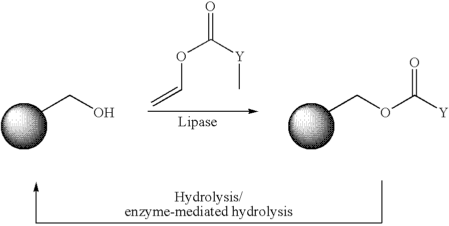

2) Acetal formation, for example, introduction of a glucuronic acid residue or a lipophile-modified version of glucuronic acid

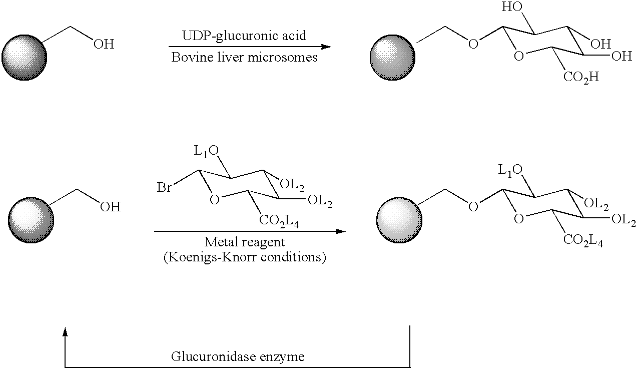

3) Phosphate formation

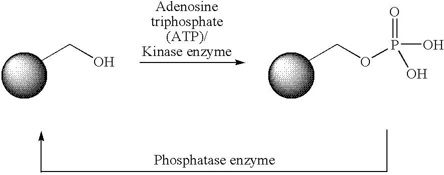

4) Phosphate diester formation

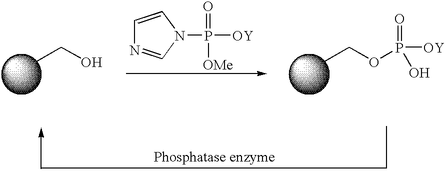

5) Oligonucleotide formation

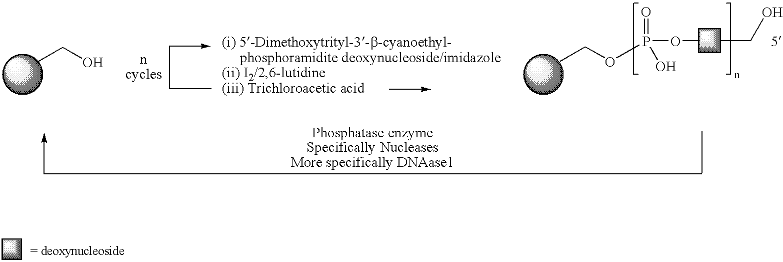

Once formed, the drug-oligonucleotide conjugate can either be base-paired with a complementary oligonucleotide according to Watson-Crick or Hoogsteen base pairing principles or can be left single stranded. The choice of whether to use double stranded, triple stranded or single stranded DNA depends on the particular phosphatase that will be used for the conversion of the prodrug into the drug.

Pharmaceutical Moieties Bearing Thiols or to Which Thiols can be Readily Introduced

1. Thioester formation 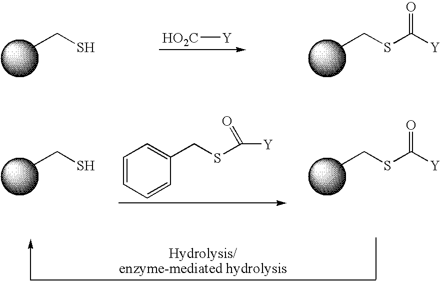
Pharmaceutical Moieties Bearing Carboxylic Acids or to Which Carboxylic Acids can be Readily Introduced

1. Amide formation

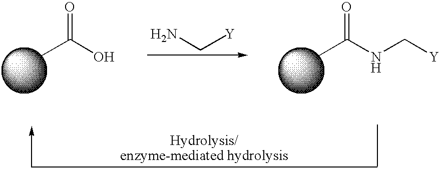

2. Amide formation (via thioesters)

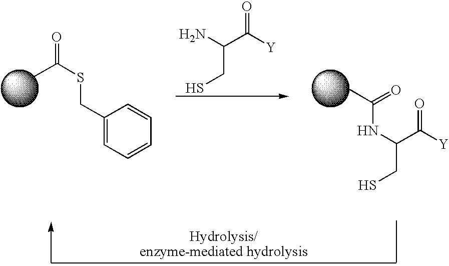

3. Ester formation 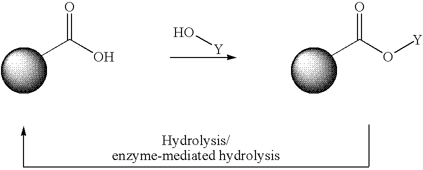
Pharmaceutical Moieties Bearing Aldehydes and Ketones or to Which Aldehydes and Ketones can be Readily Introduced

2. Oxime or hydrazone formation 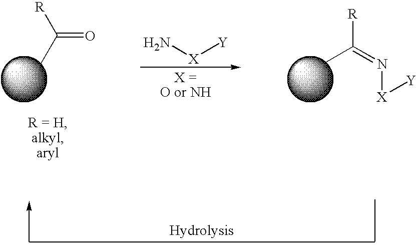
Pharmaceutical Moieties Bearing Phosphate Groups or Their Derivatives

1. Phosphate-linked dimers can be prepared as shown below.

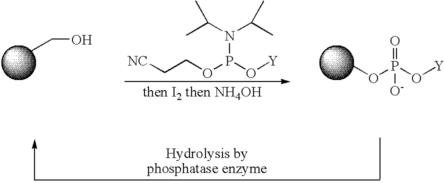

When the pharmaceutical moiety is the preferred antimicrobial agent, then modification is preferably carried out with the primary goal of increasing residence time but this may also be accompanied by an increase in potency or therapeutic index. The choice of position at which modification should be carried out should be guided by knowledge of how the antimicrobial agent is likely to be revealed by enzymes present in the subject or knowledge of the enzymes produced by the antimicrobial agent.

Pharmaceutically acceptable salts of the prodrugs of Formula (I), (II) or (III) may be prepared according to known procedures.

For use in therapy it is preferable that the prodrugs of Formula (I), (II) or (III) are in crystalline form.

The prodrugs of Formula (I), (II) or (III) depending on the nature of the pharmaceutically active moiety may possess antimicrobial activity, preferably antibacterial activity.

The term “microbial infection” is used herein in its broadest sense and refers to any infection caused by a microorganism and includes bacterial and viral infections. Examples of such infectious microorganisms may be found in a number of well known texts such as ‘Medical Microbiology’ (Greenwood, D., Slack, R., Peutherer, J., Churchill Livingstone Press, 2002); ‘Mims’ Pathogenesis of Infectious Disease' (Mims, C., Nash, A., Stephen, J., Academic Press, 2000); ‘“Fields” Virology. (Fields, B. N., Knipe, D. M., Howley, P. M., Lippincott Williams and Wilkins, 2001).

The term “microorganism” includes any microscopic organism or taxonomically related macroscopic organism within the categories algae, bacteria, fungi, protozoa, viruses and subviral agents or the like. Although, the preferable microorganism is those found in sources described above. For example, those microorganisms found in anaerobic sludge such as methanogens, eubacteria or nitrifying bacteria.

Bacterial infections include, but are not limited to, infections caused by Gram Positive Bacteria including Bacillus cereus, Bacillus anthracis, Clostridium botulinum, Clostridium difficile, Clostridium tetani, Clostridium perfringens, Corynebacteria diphtheriae, Enterococcus (Streptocodcus D), Listeria monocytogenes, Pneumoccoccal infections (Streptococcus pneumoniae), Staphylococcal infections such as Staphylococcus aureus and Streptococcal; Gram Negative Bacteria including Bacteroides, Bordetella pertussis, Brucella, Acinetobacter, Campylobacter, Citrobacter, enterohaemorrhagic Escherichia coli (EHEC/E. coli 0157: H7) enteroinvasive Escherichia coli (EIEC), Enterobacter, enterotoxigenic Escherichia coli (ETEC), Haemophilus influenzae, Helicobacter pylori, Klebsiella pneumoniae, Legionella spp., Moraxella catarrhalis, Neisseria gonnorrhoeae, Neisseria meningitidis, Proteus spp., Pseudomonas aeruginosa, Salmonella spp., Serratia, Vibrio cholera and Yersinia; acid fast bacteria including Mycobacterium tuberculosis, Mycobacterium avium-intracellulare, Myobacterium johnei, Mycobacterium leprae, atypical bacteria, Chlamydia, Mycoplasma, Rickettsia, Spirochetes, Treponema pallidum, Borrelia recurrentis, Borrelia burgdorfii and Leptospira icterohemorrhagiae; or other miscellaneous bacteria, including Actinomyces and Nocardia.

Preferably, the bacterial infection is a Gram Negative or Gram Positive infection such as infections associated with the respiratory tract (e.g. pneumonia associated with Klebsiella, mycobacterium species including tuberculosis and pseudomonas aeruginosa), urinary tract, GI tract and systemic infections caused by enteric bacteria such as Escherichia coli, Proteus, Serratia, Actineobacter, Citrobacter, Enterobacter and Staphylococcus aureus and plague.

Viral infections include, but are not limited to those caused by Adenovirus, Lassa fever virus (Arenavirus), Astrovirus, Hantavirus, Rift Valley Fever virus (Phlebovirus), Calicivirus, Ebola virus, Marburg Virus, Japanese encephalitis virus, Dengue virus, Yellow fever virus, Hepatitis C virus, Hepatitis G virus, Hepatitis B virus, Hepatitis D virus, Herpes simplex virus 1, Herpes simplex virus 2, Cytomegalovirus, Epstein Barr virus, Varicella Zoster Virus, Human Herpesvirus 7, Human Herpesvirus 8, Influenza virus, Parainfluenza virus, Rubella virus, Mumps virus, Morbillivirus, Measles virus, Respiratory Syncytial virus, Papillomaviruses, JC virus (Polyomavirus), BK virus (Polyomavirus), Parvovirus, Coxsackie virus (A and B), Hepatitis A virus, Polioviruses, Rhinoviruses, Reovirus, Rabies Virus (Lyssavirus), Human Immunodeficiency virus 1 and 2 and Human T-cell Leukemia virus.

Examples of viral infections include Adenovirus acute respiratory disease, Lassa fever, Astrovirus enteritis, Hantavirus pulmonary syndrome, Rift valley fever, Hepatitis E, diarrhoea, Ebola hemorrhagic fever, Marburg hemorrhagic fever, Japanese encephalitis, Dengue fever, Yellow fever, Hepatitis C, Hepatitis G, Hepatitis B, Hepatitis D, Cold sores, Genital sores, Cytomegalovirus infection, Mononucleosis, Chicken Pox, Shingles, Human Herpesvirus infection 7, Kaposi Sarcoma, Influenza, Brochiolitis, German measles, Mumps, Measles (rubeola), Measles, Brochiolitis, Papillomas (Warts), cervical cancer, Progressive multifocal leukoencephalopathy, Kidney disease, Erythema infectiosum, Viral myocarditis, meninigitis, entertitis, Hepititis, Poliomyelitis, Cold, Diarrhoea, Rabies, AIDS and Leukemia.

Preferably, the viral infection is an orthomyxovirus or paramyxovirus infection, for example, influenza A or B, parainfluenza, mumps or Newcastle disease. More preferably the viral infection is an influenza A or B infection.

Fungal infections include, but are not limited to, infections caused by Alternaria alternata, Aspergillus flavus, Aspergillus fumigatus, Aspergillus nidulans, Aspergillus niger, Aspergillus versicolor, Blastomyces dermatiditis, Candida albicans, Candida dubliensis, Candida krusei, Candida parapsilosis, Candida tropicalis, Candida glabrata, Coccidioides immitis, Cryptococcus neoformans, Epidermophyton floccosum, Histoplasma capsulatum, Malassezia furfur, Microsporum canis, Mucor spp., Paracoccidioides brasiliensis, Penicillium marneffei, Pityrosporum ovale, Pneumocystis carinii, Sporothrix schenkii, Trichophyton rubrum, Trichophyton interdigitale, Trichosporon beigelii and Rhodotorula spp..

Yeast infections include, but are not limited to, infections caused by Brettanomyces clausenii, Brettanomyces custerii, Brettanomyces anomalous, Brettanomyces naardenensis, Candida himilis, Candida intermedia, Candida saki, Candida solani, Candida tropicalis, Candida versatilis, Candida bechii, Candida famata, Candida lipolytica, Candida stellata, Candida vini, Debaromyces hansenii, Dekkera intermedia, Dekkera bruxellensis, Geotrichium sandidum, Hansenula fabiani, Hanseniaspora uvarum, Hansenula anomala, Hanseniaspora guillermondii Hanseniaspora vinae, Kluyveromyces lactis, Kloekera apiculata, Kluveromyces marxianus, Kluyveromyces fragilis, Metschikowia pulcherrima, Pichia guilliermodii, Pichia orientalis, Pichia fermentans, Pichia memranefaciens, Rhodotorula Saccharomyces bayanus, Saccharomyces cerevisiae, Saccharomyces dairiensis Saccharomyces exigus, Saccharomyces uinsporus, Saccharomyces uvarum, Saccharomyces oleaginosus, Saccharomyces boulardii, Saccharomycodies ludwigii, Schizosaccharomyces pombe, Torulaspora delbruekii, Torulopsis stellata, Zygoaccharomyces bailli and Zygosaccharomyces rouxii.

Protozoal infections include, but are not limited to, infections caused by Leishmania, Toxoplasma, Plasmodia, Theileria, Anaplasma, Giardia, Trichomonas, Trypanosoma, Coccidia, and Babesia. Specific examples include Trypanosoma cruzi, Eimeria tenella, Plasmodium falciparum, Plasmodium vivax or Plasmodiumn ovale.

The term “subject” as used herein refers to any animal having a disease or condition which requires treatment with a pharmaceutically-active agent. The subject may be a mammal, preferably a human, or may be a non-human primate or non-primates such as used in animal model testing. While it is particularly contemplated that the compounds of the invention are suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates.

Suitable mammals include members of the Orders Primates, Rodentia, Lagomorpha, Cetacea, Carnivora, Perissodactyla and Artiodactyla. Members of the Orders Perissodactyla and Artiodactyla are particularly preferred because of their similar biology and economic importance.

For example, Artiodactyla comprises approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghorn (Antilocapridae), and cattle, sheep, goats and antelope (Bovidae). Many of these animals are used as feed animals in various countries. More importantly, many of the economically important animals such as goats, sheep, cattle and pigs have very similar biology and share high degrees of genomic homology.

The Order Perissodactyla comprises horses and donkeys, which are both economically important and closely related. Indeed, it is well known that horses and donkeys interbreed.

As used herein, the term “effective amount” is meant an amount of the prodrug of Formula (I), (II) or (III) effective to preventing or treating a microbial infection in order to yield a desired therapeutic response. For example, to overcome or alleviate the effects of a microbial infection.

The term “therapeutically-effective amount” means an amount of the prodrug of Formula (I), (II) or (III) to yield a desired therapeutic response. For example, treating or preventing a microbial infection.

The specific “therapeutically-effective amount” will, obviously, vary with such factors as the particular microbial infection being treated, the physical condition of the subject, the type of animal being treated, the duration of the treatment, the nature of concurrent therapy (if any), and the specific formulation employed and the structure of the compound or its derivatives.

Generally, the terms “treating”, “treatment” and the like are used herein to mean affecting a subject, tissue or cell to obtain a desired pharmacologic and/or physiologic effect. The effect may be prophylactic in terms of completely or partially preventing a microbial infection or sign or symptom thereof, and/or may be therapeutic in terms of a partial or complete cure of a microbial infection. “Treating” as used herein covers any treatment of, or prevention of a microbial infection in a vertebrate, a mammal, particularly a human, and includes: (a) preventing the microbial infection from occurring in a subject that may be predisposed to the microbial infection, but has not yet been diagnosed with the microbial infection; (b) inhibiting the microbial infection, i.e., arresting its development; or (c) relieving or ameliorating the effects, i.e., cause regression of the symptoms of the microbial infection.

The prodrugs of the invention may also be used in diagnostic methods, in particular methods for the detection of microbial infections such as the influenza virus. For use in such methods it may be advantageous to link a prodrug of the invention to a label, such as a radioactive, fluorescent or chemiluminescent label.

Methods of diagnosis for which the prodrugs of the invention are suitable are described, for example, in our earlier applications PCT/AU97/00109 and PCT/AU97/00771.

It will be further appreciated that the amount of the prodrug of the invention required for use in treatment will vary not only with the particular prodrug selected but also with the route of administration, the nature of the condition being treated, and the age and condition of the subject, and will ultimately be at the discretion of the attendant physician or veterinarian. In general however, a suitable dose will be in the range of from about 0.001 to 100 mg/kg of bodyweight. per day, preferably in the range of 0.001 to 1 mg/kg/day, most preferably in the range of 0.002 to 0.1 mg/kg/day.

Treatment is preferably commenced before or at the time of infection and continued until microorganism is no longer present. However the prodrugs are also effective when given post-infection, for example, after the appearance of established symptoms.

Suitably treatment is given on one or two occasions, preferably only once only for treatment and preferably once per week for prophylaxis.

The prodrug is conveniently administered in unit dosage form, for example containing 1 to 100 mg, more conveniently 0.1 to 10 mg, most conveniently 0.1 to 5 mg of active ingredient per unit dosage form.

While it is possible that, for use in therapy, the prodrug of the invention may be administered as the raw chemical, it is preferable to present the active ingredient as a pharmaceutical formulation.

The carrier(s) must be “acceptable” in the sense of being compatible with the other ingredients of the formulation and not being deleterious to the subject thereof.

The prodrugs of the invention may also be used in combination with other therapeutic and/or prophylactic agents, for example other antimicrobial or antiinfective agents. In particular the prodrugs of the invention may be employed with other antibacterial agents.

The combinations referred to above may conveniently be presented for use in the form of a pharmaceutical formulation and thus such formulations comprising a combination as defined above together with a pharmaceutically acceptable carrier form part of the invention.

Suitable therapeutic and/or prophylactic agents for use in such combinations include other antimicrobial agents, in particular antibacterial agents such as combinations of trimethoprim and sulfonamide; bacitracin and polymyxin B-neomycin; imipenem and fluoroquinolone; and beta-lactam and aminoglycosides.

The individual components of such combinations may be administered either separately, sequentially or simultaneously in separate or combined pharmaceutical formulations.

When the prodrugs of the invention are used with a second therapeutic and/or prophylactic agent active against the same microorganism, the dose of each prodrug may either be the same as or different from that employed when each prodrug is used alone. Appropriate doses will be readily appreciated by those skilled in the art.

Pharmaceutical formulations include those suitable for oral, rectal, nasal, topical (including buccal and sub-lingual), vaginal or parenteral (including intramuscular, sub-cutaneous and intravenous) administration, or those in a form suitable for administration to the respiratory tract (including the nasal passages) for example by inhalation or insufflation. The formulations may, where appropriate, be conveniently presented in discrete dosage units, and may be prepared by any of the methods well known in the art of pharmacy. These methods include the step of bringing into association the prodrug with liquid carriers or finely divided solid carriers or both, and then, if necessary, shaping the product into the desired formulation.

Pharmaceutical formulations suitable for oral administration may conveniently be presented as discrete units such as capsules, cachets or tablets each containing a predetermined amount of the prodrug; as a powder or granules; as a solution, a suspension or as an emulsion. The prodrug may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may for example be in the form of aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles, which may include edible oils, or preservatives.

The prodrugs according to the invention may also be formulated for parenteral administration by injection, for example bolus injection, or continuous infusion, and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulating agents such as suspending, stabilising and/or dispersing agents. Alternatively, the prodrug may be in powder form, obtained by aseptic isolation of sterile solid or by lyophilisation from solution, for constitution with a suitable vehicle, eg. sterile, pyrogen-free water, before use.

For topical administration to the epidermis the prodrugs according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base, and will in general also contain one or more emulsifying agents, stabilising agents, dispersing agents, suspending agents, thickening agents, or colouring agents.

Formulations suitable for topical administration in the mouth include lozenges comprising the prodrug in a flavoured base, usually sucrose and gum acacia or gum tragacanth; pastilles comprising the prodrug in an inert base such as gelatin or sucrose and gum acacia; and mouthwashes comprising the prodrug in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the prodrug with the softened or melted carrier(s) followed by chilling and shaping moulds.

Formulations suitable for vaginal administration may be presented as pessaries, tampons, creams, gels, pastes, foams or sprays containing in addition to the active ingredient such carriers as are known in the art to be appropriate.

For administration to the respiratory tract, including intranasal administration, the neuraminidase inhibitors may be administered by any of the methods and formulations employed in the art for administration to the respiratory tract.

Thus in general the prodrugs may be administered in the form of a solution or a suspension or as a dry powder.

Solutions and suspensions will generally be aqueous, for example prepared from water alone (for example sterile or pyrogen-free water) or water and a physiologically acceptable co-solvent (for example ethanol, propylene glycol or polyethylene glycols such as PEG 400).

Such solutions or suspensions may additionally contain other excipients for example preservatives (such as benzalkonium chloride), solubilising agents/surfactants such as polysorbates (eg. Tween 80, Span 80, benzalkonium chloride), buffering agents, isotonicity-adjusting agents (for example sodium chloride), absorption enhancers and viscosity enhancers. Suspensions may additionally contain suspending agents (for example microcrystalline cellulose, carboxymethyl cellulose sodium).

Solutions or suspensions are applied directly to the nasal cavity by conventional means, for example with a dropper, pipette or spray. The formulations may be provided in single or multidose form. In the latter case a means of dose metering is desirably provided. In the case of a dropper or pipette this may be achieved by the subject administering an appropriate, predetermined volume of the solution or suspension. In the case of a spray this may be achieved for example by means of a metering atomising spray pump.

Administration to the respiratory tract may also be achieved by means of an aerosol formulation in which the compound is provided in a pressurised pack with a suitable propellant, such as a chlorofluorocarbon (CFC), for example dichlorodifluoromethane, trichlorofluoromethane or dichlorotetrafluoroethane, carbon dioxide or other suitable gas. The aerosol may conveniently also contain a surfactant such as lecithin. The dose of drug may be controlled by provision of a metered valve.

Alternatively the prodrugs may be provided in the form of a dry powder, for example a powder mix of the compound in a suitable powder base such as lactose, starch, starch derivatives such as hydroxypropylmethyl cellulose and polyvinylpyrrolidine (PVP). Conveniently the powder carrier will form a gel in the nasal cavity. The powder composition may be presented in unit dose form, for example in capsules or cartridges of eg. gelatin, or blister packs from which the powder may be administered by means of an inhaler.

In formulations intended for administration to the respiratory tract, including intranasal formulations, the prodrug will generally have a small particle size, for example of the order of 5 microns or less. Such a particle size may be obtained by means known in the art, for example by micronisation.

When desired, formulations adapted to give sustained release of the prodrug may be employed.

Preferably the prodrugs of the invention are administered to the respiratory tract by inhalation, insufflation or intranasal administration, or a combination thereof.

“Relenza” is administered by oral inhalation as a free-flow powder via a “Diskhaler” (trade mark of Glaxo Wellcome plc). A similar formulation would be suitable for the present invention.

It will be appreciated that the inhaler may also be in the form of a meter dose aerosol inhaler.

### BRIEF DESCRIPTION OF THE DRAWINGS

In the Examples, reference will be made to the accompanying drawings in which:

FIG. 1 is a graph of tobramycin concentration relative to kanamycin A 168 hours post dose;

FIG. 2 is the MS/MS spectra for compound 26;

FIG. 3 is the MS/MS spectra for compound 29; and

FIG. 4 is the MS/MS spectra for compound 30.

### EXAMPLES

The invention will now be described in detail by way of reference only to the following non-limiting examples.

The examples describe methods for the preparation of hydrolysis-activated prodrugs of aminoglycoside antibiotic tobramycin.

### Example 1

### Ester Linkage—Activation by Hydrolysis

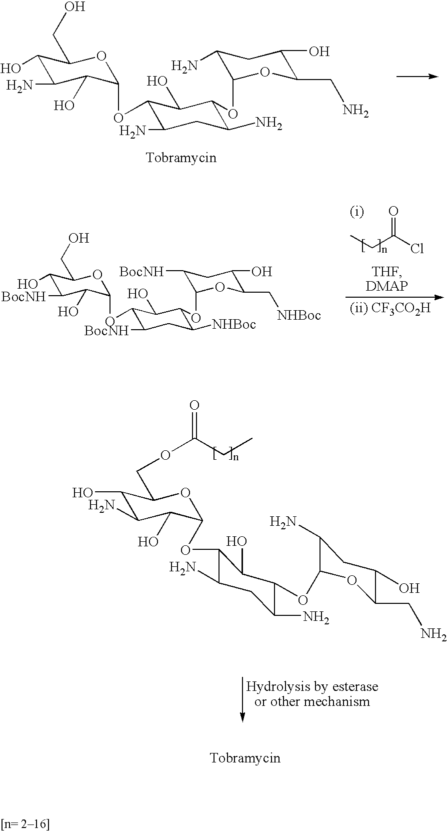

### Example 2

### Tobramycin 6″-Palmitoyl Ester

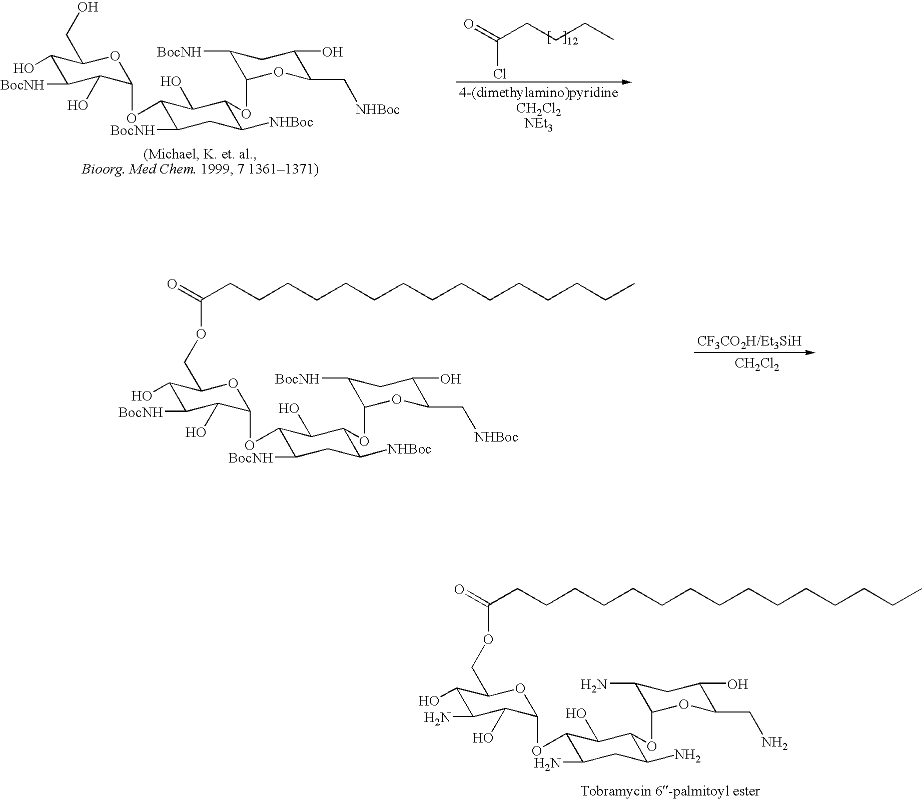

Boc-tobramycin (68 mg, 0.07 mmole) in dichloromethane (15 mL) was treated with 4-(dimethylamino)pyridine (13.2 mg, 0.108 mmole) and palmitoyl chloride (27.5 mg, 0.1 mmole). The reaction mixture was stirred at room temperature for 30 minutes then treated with triethylamine (143 μL, 1.08 mmole) and stirred for a further 18 hours at room temperature. The dichloromethane was then removed under reduced pressure and the residue dissolved in 1:1 water:diethyl ether. The ether layer was washed with water and saturated aqueous sodium bicarbonate then dried, filtered and concentrated to give 61 mg of residue.

The residue was dissolved in dichloromethane (3 mL) and then treated with triethylsilane (100 μL) and trifluoroacetic acid (3 mL). After 2 hours stirring at room temperature, the volatiles were removed under reduced pressure and the residue purified by preparative LCMS. Calculated for C₃₄H₆₇N₅O_{10,} 705. Found (ESMS) 706 ([M+H]+).

### Example 3

### Amide Linkage—Activation by Hydrolysis

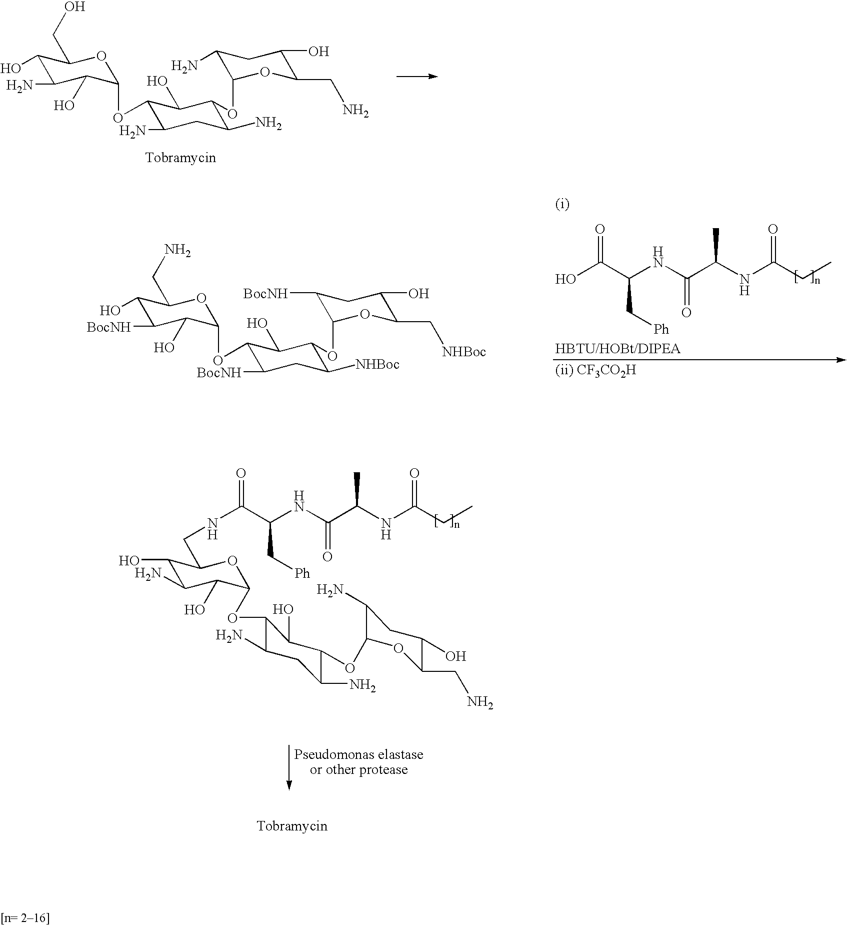

### Example 4

### Oligodeoxynucleotide Formation

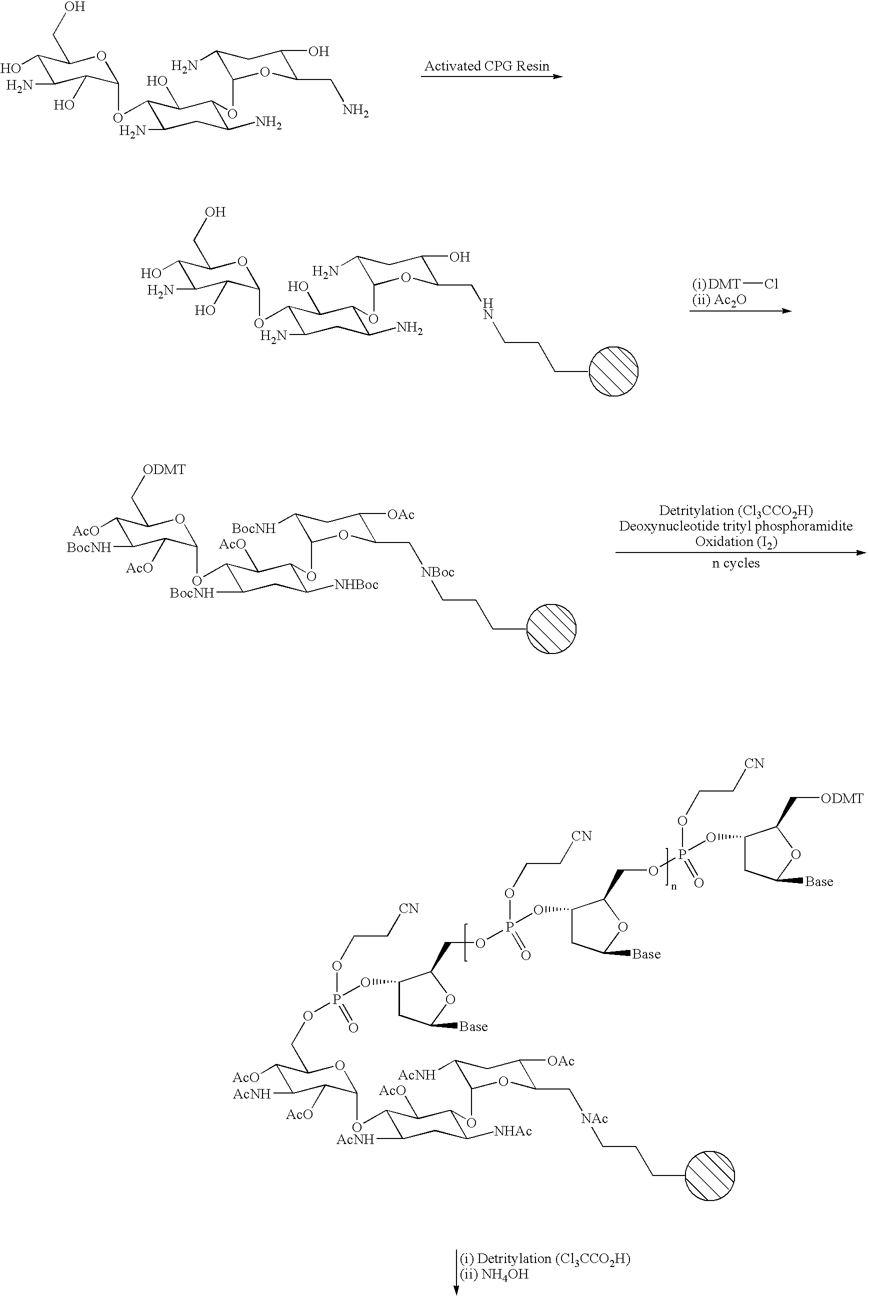

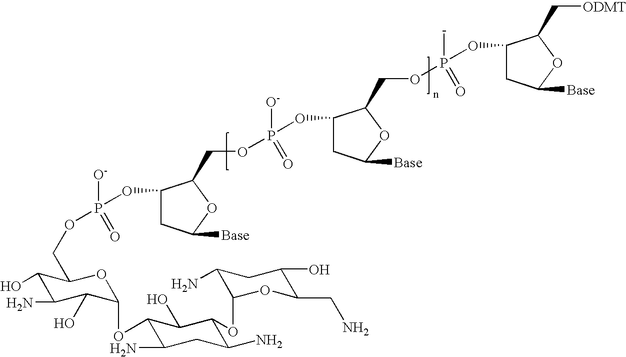

### Example 5

### Preparation of Aminoglycoside Esters, Amides and Oximes and Aminoglycoside Dimers

Aminoglycoside intermediates 1 and 2 were prepared as shown in Scheme 1 by adaptation of procedures previously published by Michael, K. et al., Bioorg. Med. Chem. 1999, 7 1361-1371. 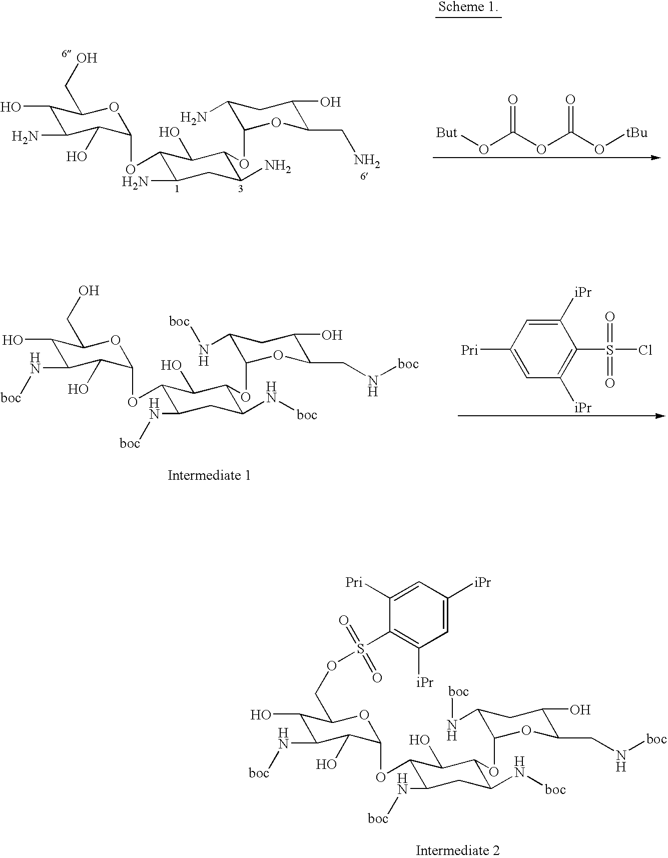
General Method for Preparation of Cesium Salts From Carboxylic Acids.
Intermediates 3 were prepared by a modified method previously described by J. Org. Chem, 1987, 52, 4230-4234 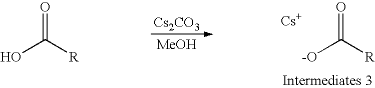
To a solution of cesium carbonate (3 mmol) in anhydrous MeOH (20 mL) was added a solution of carboxylic acid (9 mmol) in MeOH (9 mL). The mixture was stirred under argon for 30 mins and solvent evaporated. The resulting white solid was washed thoroughly with ether to remove any un-reacted acid and the solid material was dried under vacuum (1 mmHg) at 80° C. over P₂O₅ then stored under dry nitrogen.
Method A:

To a solution consisting of the appropriate intermediate 3 (248 μmol) in anhydrous DMF (5 mL) was added a solution of intermediate 2 (40.5 μmol) in anhydrous DMF (2 mL). The mixture was stirred at 75° C. for 24 h under a dry nitrogen atmosphere. The reaction was monitored by LCMS. Upon completion of the reaction, the solvent was removed in vacuo to leave a sticky residue, which was re-dissolved in CF₃CO₂H (2 mL) at room temperature. After 5 mins the CF₃CO₂H was removed under a stream of nitrogen, the residues were re-dissolved in H₂O (10 mL) and the solution lyophilized. The product was isolated by reverse phase chromatography, conditions for which are provided in Table 5.
Method B:

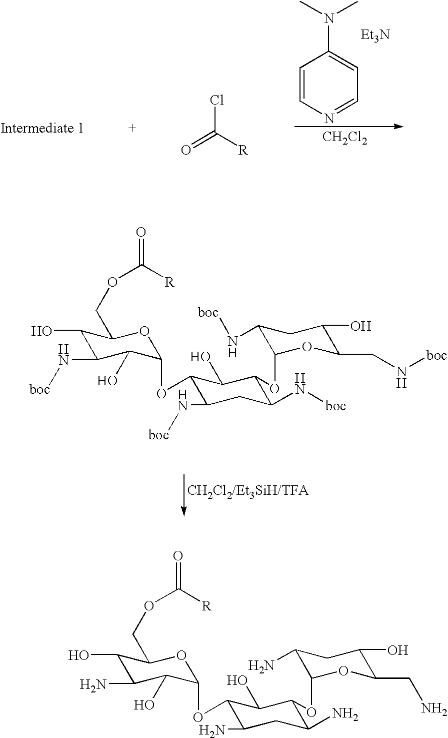

Intermediate 1 (0.07 mmole) was dissolved in dry dichloromethane (15 ml) and N,N-dimethylaminopyridine (DMAP) (0.108 mmole) was added followed by the appropriate acid chloride (0.1 mmole). After stirring at ambient temperature for 30 min., triethylamine (0.108 mmole) was added and the reaction was stirred for 16 h at room temperature. The solvent was removed under vacuum and the residue was partitioned between a mixture of ether (10 ml) and water (10 ml). The ether phase was separated, washed with water, then saturated sodium bicarbonate solution, dried over anhydrous sodium sulphate and concentrated in vacuo.

The crude residue was dissolved in dichloromethane (3 ml) and triethylsilane (TES) (100 μl) was added followed by trifluoroacetic acid (3 ml). The solution was stirred for 2 h at room temperature then evaporated to dryness under vacuum at 40° C. The crude product was purified by reverse phase chromatography.
Intermediate 4: 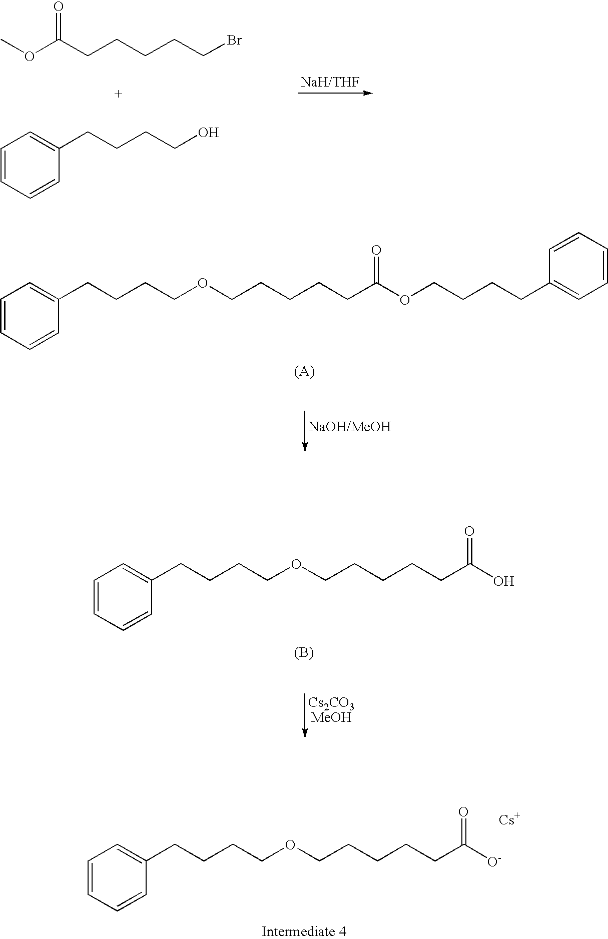
NaH (60% dispersion in oil; 6.37 mmol) was added portions wise to a solution of 4-phenyl-1-butanol (5.1 mmol) and 6-bromohexanoate (5.1 mmol) in anhydrous THF (10 mL) under an inert atmosphere. The suspension was stirred and heated on reflux for 27 h at 70° C. To the reaction mixture was cooled then treated with H₂O (20 mL) then extracted with diethyl ether (20 mL×3). The combined organic extracts were dried over MgSO₄, filtered then concentrated to dryness to obtain 6-(4-phenyl-butoxy)-hexanoic acid 4-phenyl-butyl ester (A) as an orange oil. Calculated for compound (A) C₂₆H₃₆O₃ 396; found m/z=396.91 [M+H]¹⁺. This material was dissolved in MeOH (1 mL) and 1N NaOH (1 mL) and stirred at room temperature. After 4 h, the reaction mixture was concentrated to dryness to obtain a colourless residue. 6-(4-Phenyl-butoxy)-hexanoic acid (B) was isolated by partitioning the residues between H₂O (50 mL) and CHCl₃ (50 mL). The organic layer was removed and the aqueous layer washed with two more aliquots of CHCl₃ (50 mL). The aqueous layer was lyophilized to produce a white solid. Calculated for compound (B) C₁₆H₂₄O₃ 264; found m/z 264.37 [M+H]¹⁺. Formation of the cesium salt (Intermediate 4) was performed using the generalised procedure described for intermediates 3.

Compound 14 was prepared by adopting Method A.

It would be reasonable to expect that the above methodology used to introduce esters to the 6″-position of tobramycin could also be extrapolated to other aminoglycosides equipped with appropriate leaving groups as suitable positions. For example, Michael and co-workers referenced to above have shown that leaving groups can be introduced to each of neomycin, kanamycin and tobramycin and these displaced with suitable nucleophiles. Method A described above could also be applied to these aminoglycosides. 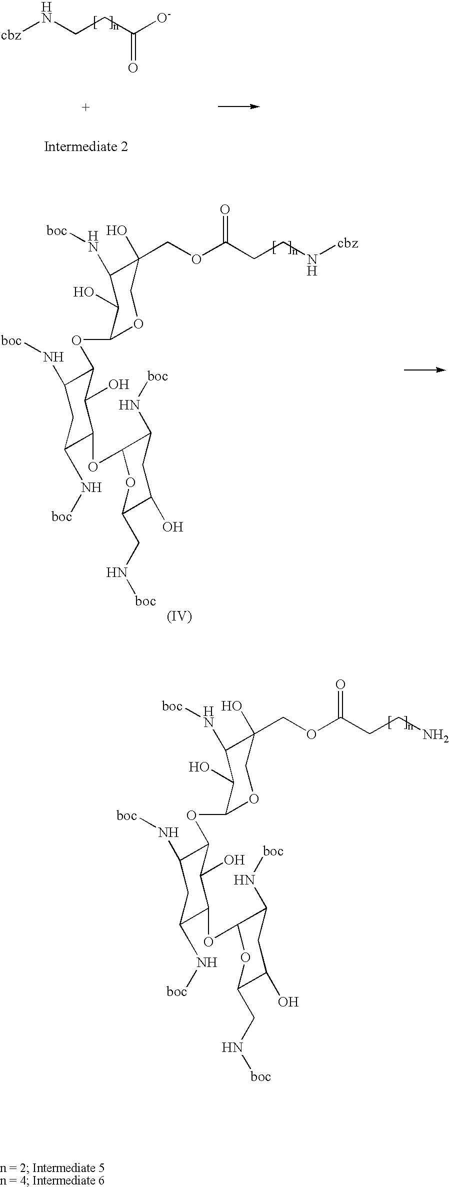
Compounds of Formula (IV) were prepared as described in Method A. The products were dissolved in minimum amount of MeOH and was purified by flash chromatography (SiO₂, 2.5% MeOH in CH₂Cl₂) prior to removal of the Cbz group. The purified material was treated with MeOH and 10% Pd/C then stirred at room temperature under hydrogen gas at atmospheric pressure for 16 h. The solvent was then removed under reduced pressure to afford intermediate 5 or 6 as white powders.
Method C:

A solution containing intermediate 5 or 6 (0.093 mmol), diacid (0.038 mmol) and benzotriazol-1-yloxytri(dimethylamino)phosphonium (BOP) (0.093 mmol) in dried N,N-dimethylacetamide (DMA) (2.5 mL) was stirred for 5 mins prior to addition of diisopropylethylamine (DIEA) (0.76 mmol). The reaction mixture was heated at 75° C. for 24 h under nitrogen. The reaction was monitored by LCMS and upon completion, the solvent was removed in vacuo to afford a dark residue. The resulting material was treated with CF₃CO₂H (2 mL) at room temperature and after 5 mins the solvent was evaporated generally to afford an oil. The product was isolated by preparative reverse phase chromatography, conditions for which are provided in Table 5.

Method D:

To a solution of intermediate 5 or 6 (0.093 mmol) and DMAP (0.093 mmol) in anhydrous DMA (2 mL) was added the appropriate diisocyanate (0.038 mmol). The mixture was allowed to stir for 48 h under nitrogen at 75° C. The solvent was removed in vacuo and the residue was treated with CF₃CO₂H (5 mL) for 5 mins. The CF₃CO₂H was removed to obtain a residue which was redissolved in H₂O (10 mL) and freeze-dried to obtain crude product. The product was purified by preparative reverse phase chromatography, conditions for which are described in Table 5.

Method E:

To a chilled solution of BOC-L-Asp(OtBu)-OH. DCHA (0.178 mmol) and BOP (0.214 mmol) in DMF (1 mL) was added a solution of tobramycin (0.214 mmol) in H₂O (2 mL). The mixture was stirred at room temperature under nitrogen for 16 h and solvent removed under vacuum to afford a colourless oil. The crude material was isolated by preparative reverse phase HPLC and the purified material was treated with 50% TFA/CH₂Cl₂ (2 mL) and 5% Et₃SiH (100 μL) for 4 h. The solvent was removed in vacuo and the residue was dissolved in H₂O (10 mL) then lyophilized overnight to afford a sandy solid.

Method F:

The di-cesium salt was prepared by the method described for intermediate 3. This intermediate (0.05 mmole) was added to a solution of intermediate 2 (0.1 mmole) in DMA (3 ml). The reaction was stirred and heated at 80° C. for 72 h under argon. The residue was triturated with water and filtered. After washing with more water, the residue was dissolved in CH₂Cl₂, dried over Na₂SO₄ and the solution evaporated to dryness.

The residue was dissolved in CF₃CO₂H (8 mL), stirred for 5 minutes and evaporated to dryness under vacuum with the minimum of heat. The product was then purified by preparative reverse phase chromatography.
General Method for Preparation of Tobramycin Oximes at the 6″-Position.

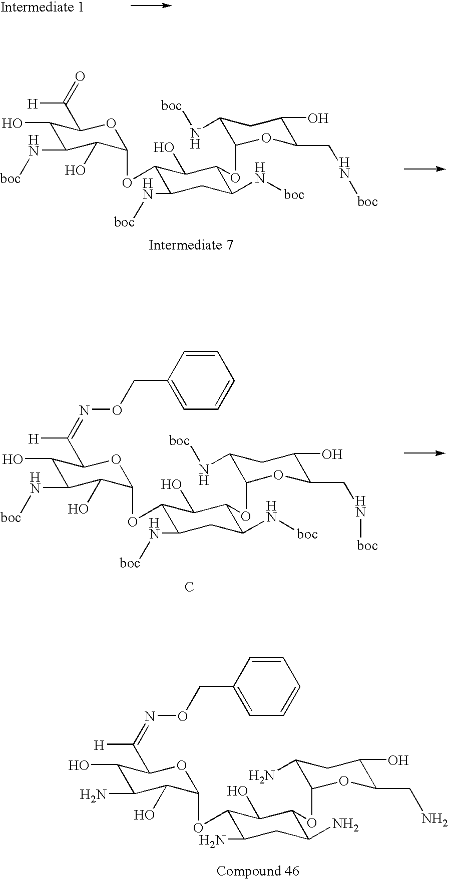

Intermediate 1 (0.052 mmole) was stirred in dry CH₂Cl₂ (2 ml) and tetrahydrofuran (2 ml) at 0° C. before the addition of Dess-Martin Periodinane (0.077 mmole). The reaction was allowed to warm to room temperature over 2 hours and concentrated in vacuo. The crude product was purified by chromatography on silica eluting with 10% MeOH in CH₂Cl₂. Rf=0.47 (10% MeOH/CH₂Cl₂). Calculated for Intermediate 7 C₄₃H₇₅N₅O₁₉ 965; found m/z 866.15 [(M-boc)+H]¹⁺.

Intermediate 7 (0.050 mmole) and O-benzylhydroxylamine hydrochloride salt (0.2 mmole) were stirred in a biphasic system with CH₂Cl₂ (3 ml) and 10 mM ammonium acetate (3 ml). After 2 days the CH₂Cl₂ layer was separated and the aqueous phase extracted with CH₂Cl₂ (3×10 ml). The combined organic layers were dried (MgSO₄), filtered and concentrated in vacuo.

The crude residue was dissolved in CH₂Cl₂ (2 ml) followed by trifluoroacetic acid (2 ml). The solution was stirred for 1 h at room temperature, evaporated to dryness in vacuo and triturated with hexane/CH₂Cl₂ (1:1 2×10 ml). The crude product was purified by reverse phase chromatography.

TABLE 2
Synthetic & Structural Details for Aminoglycoside Monoesters

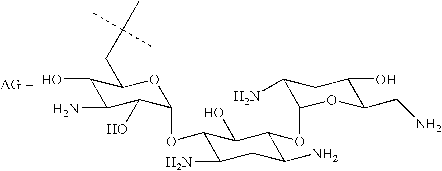

| | | Amino- | | |
|---|---|---|---|---|
| Com- | Modification reagent | glycoside | General | |
| pound | (Intermediates 3) | Intermediate | Method | |
| 1 | Cesium 1,16- hexadecanedioate | 2 | A | 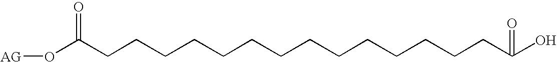 |
| 2 | Cesium 3- benzoylpropionate | 2 | A | 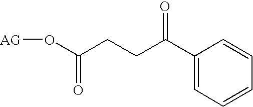 |
| 3 | Cesium 4- benzyloxybenzoate | 2 | A | 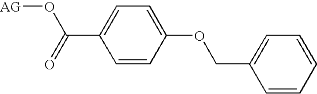 |
| 4 | Cesium phenylacetate | 2 | A | 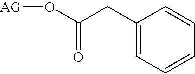 |
| 5 | Cesium 4- phenoxylbenzoate | 2 | A | 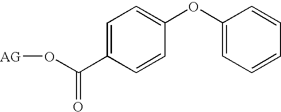 |
| 6 | Cesium trans- cinnamate | 2 | A | 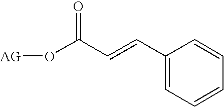 |
| 7 | Cesium benzoate | 2 | A | 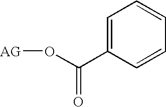 |
| 8 | Cesium BOC-amino- 3-hydroxylbutanoate | 2 | A | 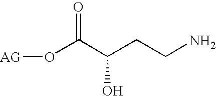 |
| 9 | Cesium 4- methoxybenzoate | 2 | A | 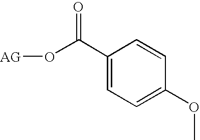 |
| 10 | Cesium 2,4,6- trimethylbenzoate | 2 | A | 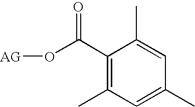 |
| 11 | Cesium 4- (trifluoromethoxy) benzoate | 2 | A | 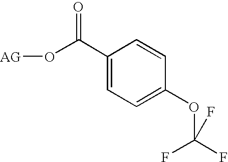 |
| 12 | Cesium 4- fluorophenylacetate | 2 | A | 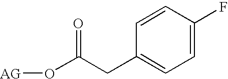 |
| 13 | Cesium 4- methoxylphenyl acetate | 2 | A | 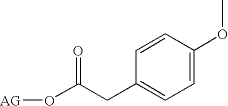 |
| 14 | Cesium 6-(4-phenyl- butoxy)-hexanoate (Intermediate 4) | 2 | A | 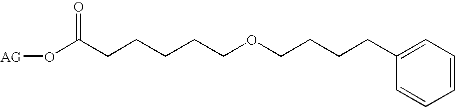 |
| 15 | Cesium isonicotinate | 2 | A | 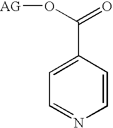 |
| 16 | Cesium indole-6- carboxylate | 2 | A | 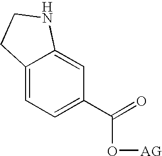 |
| 17 | Cesium indole-6- carboxylate | 2 | A | 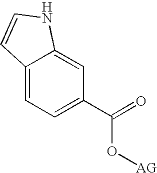 |
| 18 | Cesium 5- benzimidazole carboxylate | 2 | A | 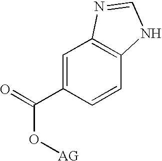 |
| 19 | Cesium indole-5- carboxylate | 2 | A | 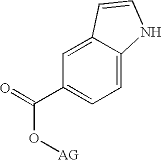 |
| 20 | Cesium salicylate | 2 | A | 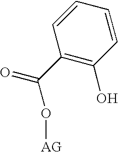 |
| 21 (Inter- med- iate 6) | Cesium Cbz- aminocaproate | 2 | A | 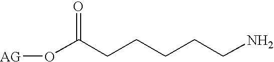 |
| 22 | Cesium terephthalate | 6 | C | 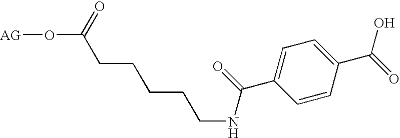 |
| 23 | Cesium butyrate | 2 | A | 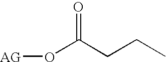 |
| 24 | Cesium heptadecanoate | 2 | A | 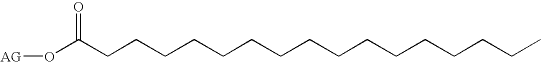 |
| 25 | Palmitoyl chloride | 1 | B | 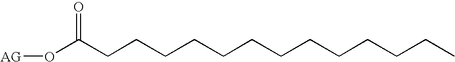 |
| 26 | Cesium dodecanoate | 2 | A | 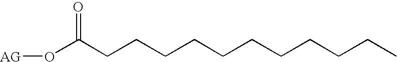 |
| 27 | Cesium decanoate | 2 | A | 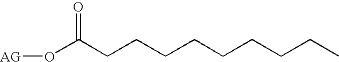 |
| 28 | Cesium octanoate | 2 | A | 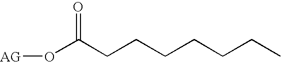 |
| 40 | Cesium 5-Bromo- furan-2-carboxylate | 2 | A | 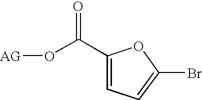 |
| 41 | Cesium 5-Nitro-1H- pyrazole-3-carboxyate | 2 | A | 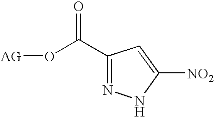 |
| 42 | Cesium 2-Methyl- furan-3-carboxylate | 2 | A | 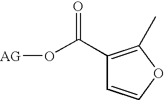 |
| 43 | Cesium 3-Hydroxy- isoxazole-5- carboxylate | 2 | A | 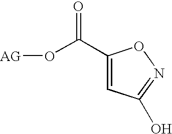 |
| 44 | Cesium Cyclohexane carboxylate | 2 | A | 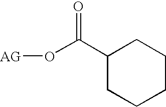 |
| 45 | Cesium Thiophene-2- carboxylate | 2 | A | 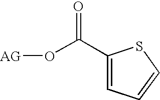 |

TABLE 3
Synthetic & Structural Details for Aminoglycoside Esters, Amides and Oximes

| | | Amino- | | |
|---|---|---|---|---|
| | | glyco- | | |
| | Modifi- | side | | |
| Com- | cation | Inter- | General | |
| pound | reagent | mediate | Method | Compound Structure |
| 29 | Palmitoyl chloride | 1 | B | 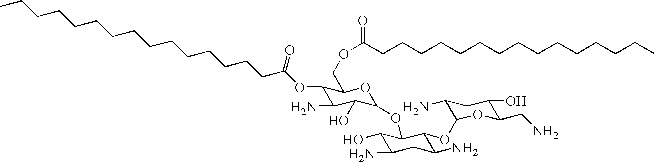 |
| 30 | Dodec- anoyl chloride | 1 | F | 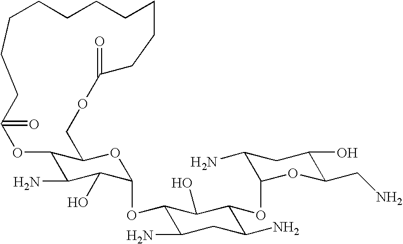 |
| 31 | Heptanoyl chloride | 1 | B | 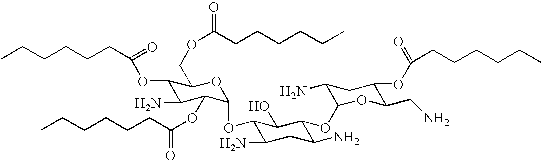 |
| 46 | O- Benzyl- hydroxyla- mine | 7 | N/A | 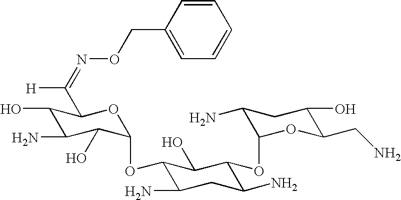 |
| 32 | Obu^{t}[BOC- L-Asp- OtBu] | Tobra- mycin | E | 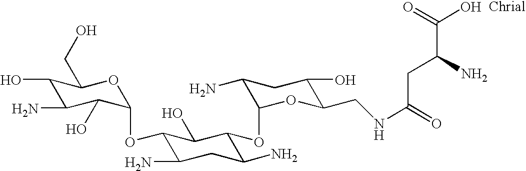 |
| 33 | BOC-L- Asp (OtBu)- OH . . . DCHA | Tobra- mycin | E | 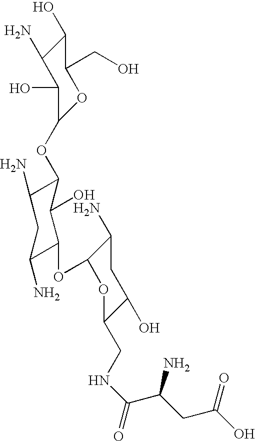 |

TABLE 4
Synthetic & Structural Details for Dimeric Aminoglycosides

| | | Amino- | | |
|---|---|---|---|---|
| | | glycoside | General | |
| Compound | Modification reagent | Intermediate | Method | Compound Structure |
| 34 | 1,4-Phenyldiisocyanate | 5 | D | 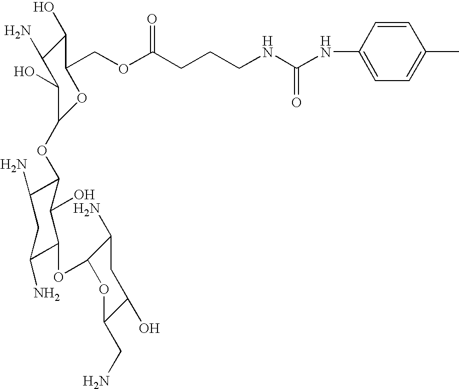 |
| | | | | 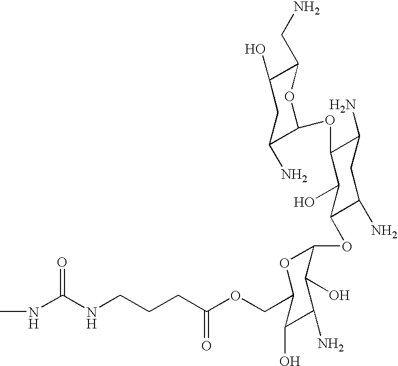 |
| 35 | Methylenebis(phenylis- ocyanate) | 5 | D | 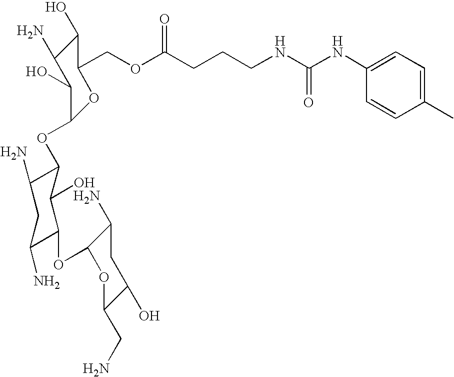 |
| | | | | 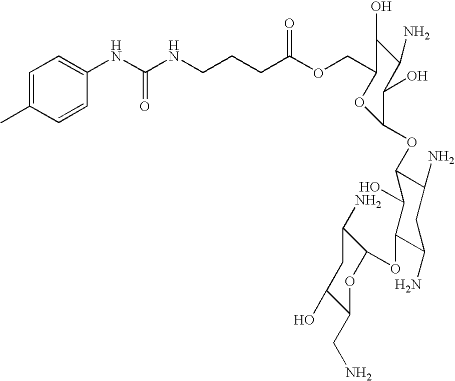 |
| 36 | Diphenic acid | 6 | C | 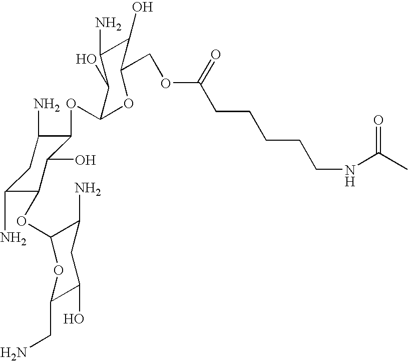 |
| | | | | 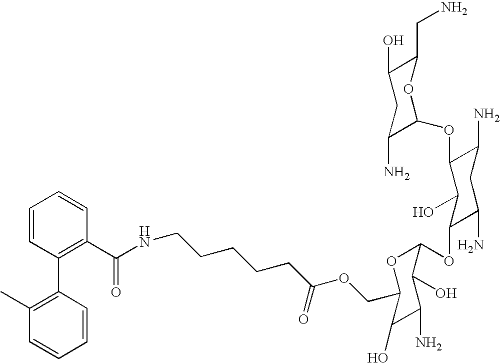 |
| 37 | 2,4,6-Trimethyl-1,3- phenylenediisocyanate | 6 | D | 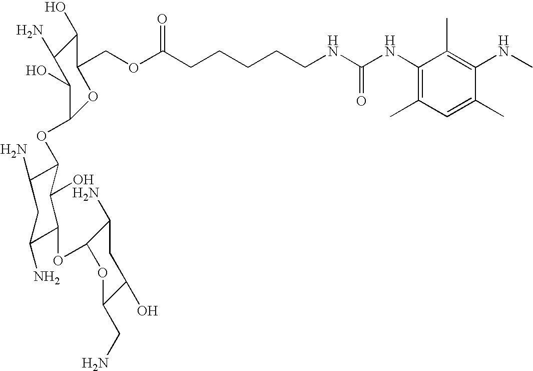 |
| | | | | 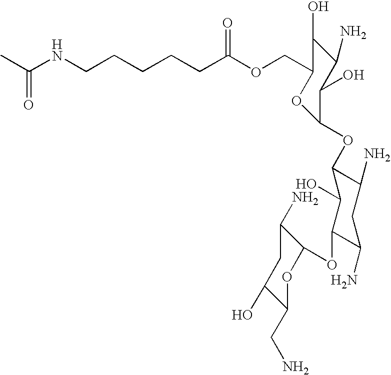 |
| 38 | Terephthalic acid | 6 | C | 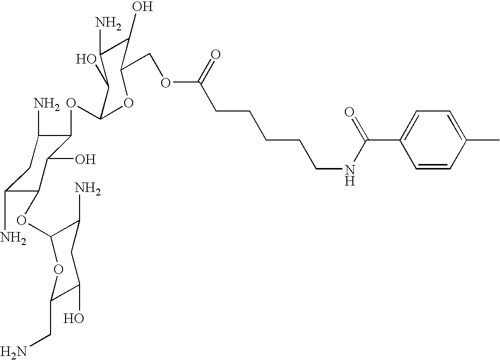 |
| | | | | 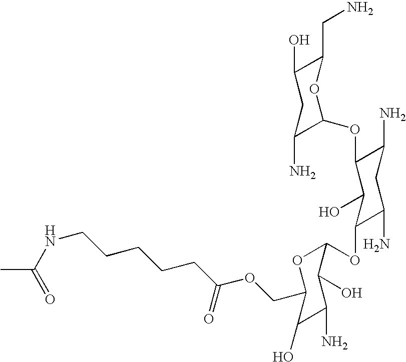 |
| 39 | Cesium 1,16- hexadecanedloate | 2 | A | 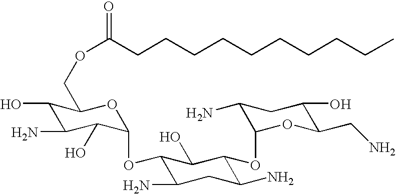 |

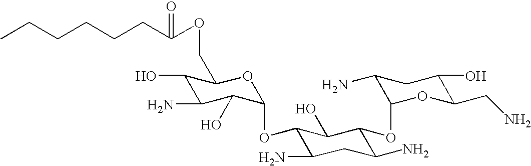

TABLE 5

| | | | | |
|---|---|---|---|---|
| | | | HPLC | GRADLENT/HPLC Method |
| Compound | Calculated | | Retention | Initial % B-Final |
| ID | MW | Observed m/z | time (min) | % B_Duration |

Characterization data for compoundB 1-20

| | | | | |
|---|---|---|---|---|
| 1 | 735.92 | 736.2[M + H]⁺ | 2.32 | GREEN; |
| | | | | 5-100_1.1 mins |
| 2 | 627.70 | 628.1[M + H]⁺ | 24.4 | BLUE: |
| | | | | 5-40_28 mins |
| 3 | 677.75 | 678[M + H]⁺ | 26.5 | BLUE; |
| | | | | 5-40_28 min |
| 4 | 585.66 | 586.14[M + H]⁺ | 21 | BLUE; |
| | | | | 5-40_28 min |
| 5 | 663.73 | 664[M + H]⁺ | 26.25 | BLUE; |
| | | | | 5-40_28 min |
| 6 | 597.67 | 598[M + 1H]¹⁺ | 22.75 | BLUE; |
| | | | | 5-40_28 min |
| 7 | 571.63 | 572.05[M + 1H]¹⁺ | 20.05 | BLUE; |
| | | | | 5-30_28 min |
| 8 | 568.63 | 569.1[M + 1H]¹⁺ | 2.65 | GREEN; |
| | | | | 5-100_1.1 mins |
| 9 | 601.66 | 602[M + 1H]¹⁺ | 21.5 | BLUE; |
| | | | | 5-40_28 mins |
| 10 | 613.71 | 614[M + 1H]¹⁺ | 28.75 | BLUE; |
| | | | | 5-20_28 mins |
| 11 | 655.63 | 655.9[M + 1H]¹⁺ | 28.5 | BLUE; |
| | | | | 5-20_28 mins |
| 12 | 603.65 | 604[M + 1H]¹⁺ | 20.5 | BLUE: |
| | | | | 10-30_28 mins |
| 13 | 615.68 | 616[M + 1H]¹⁺ | 18.75 | BLUE: |
| | | | | 10-40_28 mins |
| 14 | 713.87 | 714[M + 1H]¹⁺ | 31 | BLUE; |
| | | | | 10-40_28 mins |
| 15 | 572.62 | 572.9[M + 1H]¹⁺ | 21 | BLUE; |
| | | | | 0-20%_28 mins |
| 16 | 612.68 | 613.03[M + 1H]¹⁺ | 11.2 | BLUE; |
| | | | | 5-40_28 mins |
| 17 | 610.67 | 611.13[M + 1H]¹⁺; | 20.3 | BLUE; |
| | | 1221.25[2M + H]¹⁺ | | 5-30_28 mins |
| 18 | 611.65 | 611.9[M + 1H]¹⁺ | 15.5 | BLUE; |
| | | | | 5-15_28 mins |
| 19 | 610.67 | 611.05[M + 1H]¹⁺; | 26.5 | BLUE; |
| | | 1221.28[2M + H]¹⁺ | | 5-20_28 mins |
| 20 | 587.63 | 587.95[M + 1H]¹⁺; | 23 | BLUE; |
| | | 1174.3[2M + H]¹⁺ | | 5-30_28 mins |
| 21 | 580.68 | 581.1[M + 1H]¹⁺ | 10.75 | BLUE; |
| | | | | 0-20_28 mins |
| 22 | 728.80 | 729.1[M + 1H]¹⁺ | 22 | BLUE; |
| | | | | 10-20_28 mins |
| 23 | 537.61 | 538[M + H]⁺; | 0.92 | GREEN; |
| | | | | 5-100_1.1 mins |
| 24 | 720 | 720.2[M + 1H]¹⁺ | 23.3 | BLUE; |
| | | | | 5-80_28 min |
| 25 | 705.94 | 706.2[M + H]⁺ | 2.43 | GREEN; |
| | | | | 5-100_1.1 mins |
| 29 | 944 | 945[M + H]⁺; | NA | NA |
| | | 472.5 [M + 2H]²⁺ | | |
| 30 | 661.80 | 662.66[M + H]⁺ | 29.54 | RED |
| | | | | 20-50_20 mins |
| 31 | 916 | 916.74[M + H]⁺ | 30.5 | BLUE; |
| | | | | 15-50_28 min |
| 32 | 583 | 583.05[M + H]⁺ | 9.7 | BLUE; |
| | | | | 0-10_28 min |
| 33 | 583 | 583.11[M + H]⁺ | 0.57 | GREEN; |
| | | | | 5-100_1.1 mins |
| 34 | 1265.39 | 1265.3[M + 1H]¹⁺; | 13 | BLUE; |
| | | 633.4[M + 2H]²⁺ | | 10-15_28 mins |
| 35 | 1355.52 | 1355.2[M + 1H]¹⁺; | 23 | BLUE; |
| | | 678.3[M + 2H]²⁺ | | 10-25_28 mins |
| 36 | 1367.57 | 1367.3[M + 1H]¹⁺; | 16.5 | BLUE; |
| | | 684.5[M + 2H]²⁺ | | 10-40_28 mins |
| 37 | 1363.58 | 1363.3[M + 1H]¹⁺ | 21.25 | BLUE; |
| | | 682.5[M + 2H]²⁺ | | 5-30_28 mins |
| 38 | 1291.41 | 1291.3[M + 1H]¹⁺; | 16.5 | BLUE; |
| | | 646.5[M + 2H]²⁺ | | 0-30_28 mins |
| 39 | 1185.43 | 1185.5[M + 1H]¹⁺ | 19.2 | BLUE; |
| | | 593.4[M + 2H]²⁺ | | 5-80_28 mins |
| 40 | 640 | 639.99[M + 1H]¹⁺ | ND | — |
| | | 641.94[M + 1H]¹⁺ | | |
| 41 | 606 | 607.09 [M + 1H]¹⁺ | ND | — |
| 42 | 575 | 576.08 [M + 1H]¹⁺ | ND | — |
| 43 | 578 | 579.11 [M + 1H]¹⁺ | ND | — |
| 44 | 577 | 578.15 [M + 1H]¹⁺ | ND | — |
| 45 | 577 | 578.09 [M + 1H]¹⁺ | ND | — |
| 46 | 570 | 570.97 [M + 1H]¹⁺ | 23 | BLUE; |
| | | | | 5-25_28 mins |

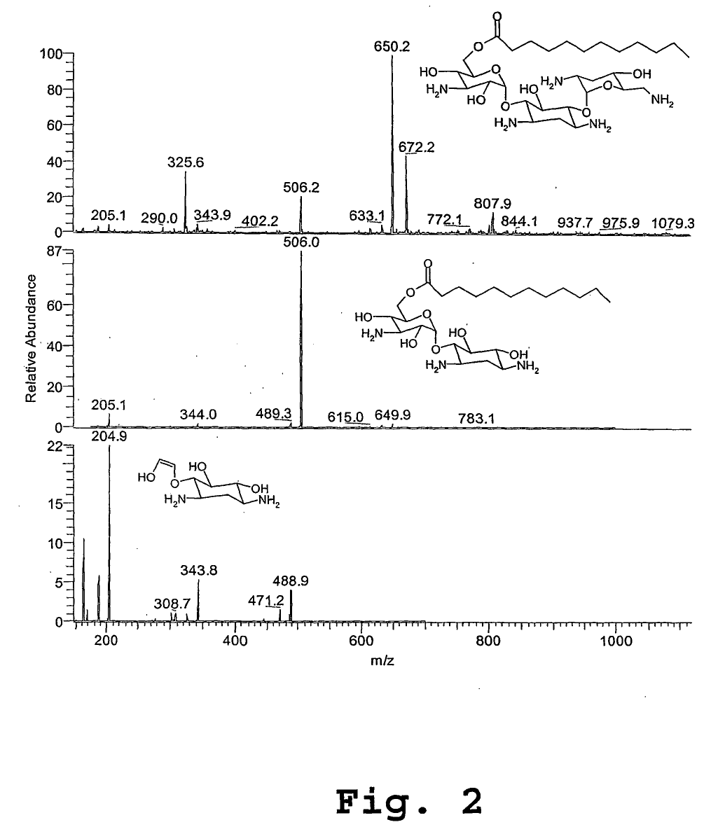

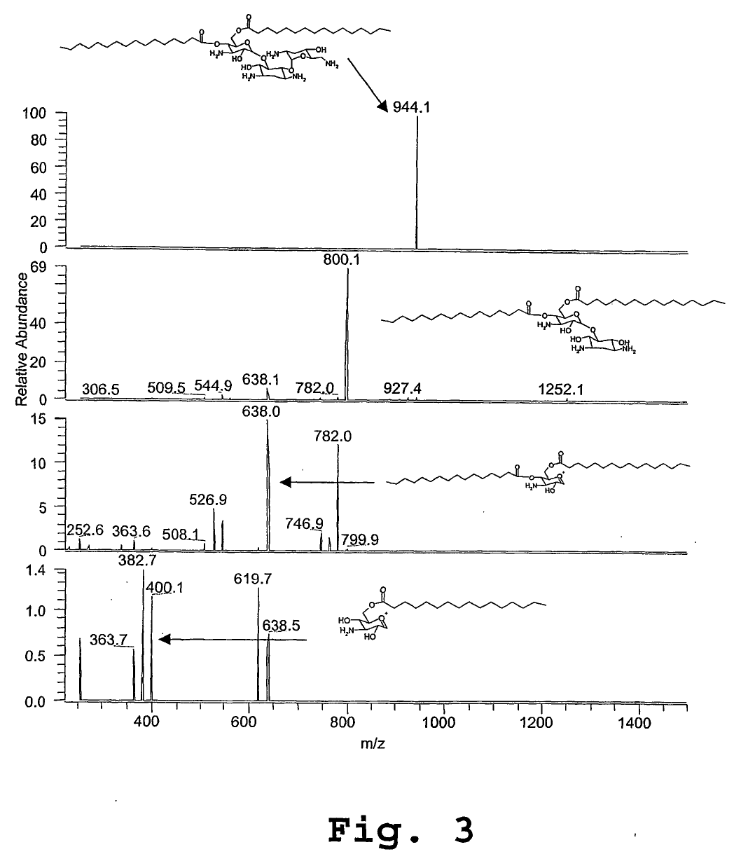

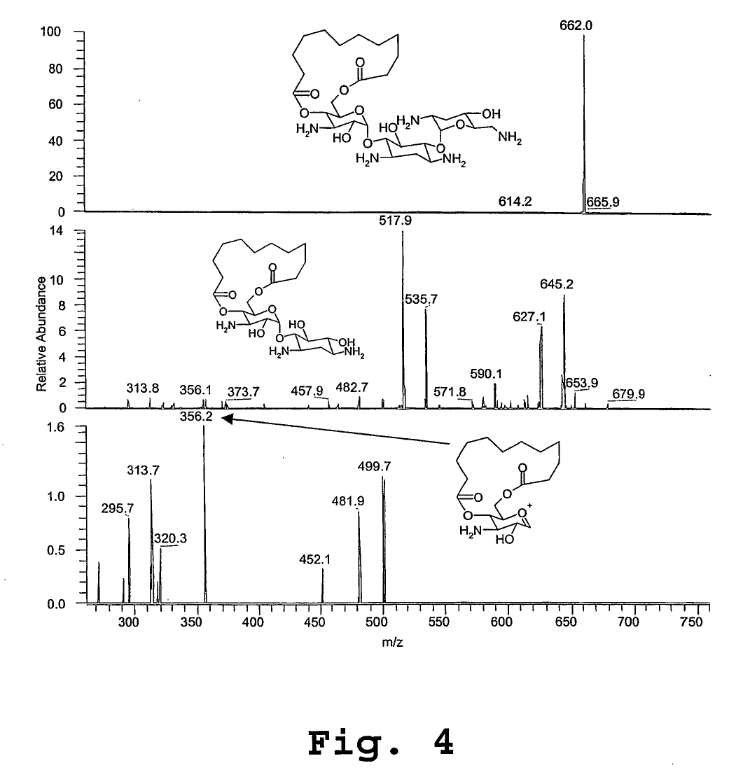

The structural characterisation of compounds 26, 29 and 30 by MS/MS using a Finnigan ion trap mass spectrometer is shown in FIGS. 2 to 4.

Preparative RP-HPLC Conditions

| | | |
|---|---|---|
| Method GREEN | Method RED | Method BLUE |

Column support and dimensions

| | Phenomenex | Phenomenex | Phenomenex |
|---|---|---|---|
| | 3μC8 SiO₂ | 10μC8 SiO₂ | 10μC8 SiO₂ |
| | (20 mm × 4 mm) | (25 cm × 2.12 cm) | (25 cm × 2.12 cm) |
| Flowrate | 1 ml/min | 10 ml/min | 10 ml/min |
| Solvent A | 0.1% TFA/H₂O | 0.1% TFA/H₂O | 0.1% TFA/H₂O |
| Solvent B | 0.06% TFA/CH₃CN | 0.06% TFA/CH₃CN | 0.06% TFA/CH₃CN |

| Gradient | Time | | | Time | | Time | |
|---|---|---|---|---|---|---|---|
| Method | (Min) | A(%) | B(%) | (Min) | B(%) | (Min) | B(%) |
| | 0 | 95 | 5 | 0 | Initial* | 0 | Initial* |
| | 0.5 | 95 | 5 | 15 | Initial* | 2 | Initial* |
| | 1.60 | 0 | 100 | 35 | Final* | 30 | Final |
| | 2.10 | 0 | 100 | 39 | 100 | 40 | 100 |
| | 2.30 | 95 | 5 | 42 | 100 | 45 | 100 |
| | 5 | 95 | 5 | 45 | Initial* | 47 | Initial* |

*For gradient information refer to Table 5.

### Example 6

### Antibiotic Activities and in Vitro Translation Inhibition of Aminoglycoside Prodrugs

Antibiotic activities were determined using measurement of minimum inhibitory concentrations by standard procedures referring to the Manual of Commercial Methods in Clinical Microbiology. Ed. Allan L. Truant. ASM Press (2002) and are reported as concentrations (μM).

| | MIC P.aeruginosa | MIC E.coli ATCC25922 |
|---|---|---|
| | ATCC27853 (μM) | (μM) |
| Tobramycin | 2 | — |
| Amikacin | 7.5 | — |
| Neomycin | 30 | — |
| Kanamycin A | 30 | — |
| 1 | 8 | 16 |
| 2 | 8 | — |
| 3 | >64 | — |
| 4 | 16 | — |
| 5 | 32 | — |
| 6 | 64 | — |
| 7 | 64 | — |
| 8 | 8 | — |
| 9 | >64 | — |
| 10 | >64 | — |
| 11 | 32 | — |
| 12 | 4 | — |
| 13 | 2 | — |
| 14 | >32 | >32 |
| 15 | 4 | 4 |
| 16 | 16 | 16 |
| 17 | 16 | >32 |
| 18 | 16 | 16 |
| 19 | 32 | >32 |
| 20 | 16 | 16 |
| 21 | 8 | 16 |
| 22 | 32 | 32 |
| 23 | 32 | — |
| 24 | 16 | — |
| 25 | 16 | — |
| 29 | 64 | — |
| 30 | 64 | — |
| 31 | 32 | — |
| 32 | >64 | — |
| 33 | >64 | >32 |
| 34 | 8 | 8 |
| 35 | 4 | 8 |
| 36 | 8 | 8 |
| 37 | 16 | 16 |
| 38 | 32 | 16 |
| 39 | 4 | — |

### Example 7

### Assessment of Enhanced Residency Time of Aminoglycoside Prodrugs

Rodents were anaesthetized with Ketamine/Domitor mixture according to standard procedures⁶ and dosed by the intra-nasal route with a solution containing an equimolar quantity of the compound of interest and kanamycinA (as internal standard) at a dose volume of approximately 3.0 ml/kg. The rodent was held in the vertical position during dosing of 30 μL per nostril. At 168 hours post-dose, mouse lungs were harvested and levels of compound in the lung tissue were assessed by analytical methods. Any analytical method suitable for detection of this type of compound may be used⁷.

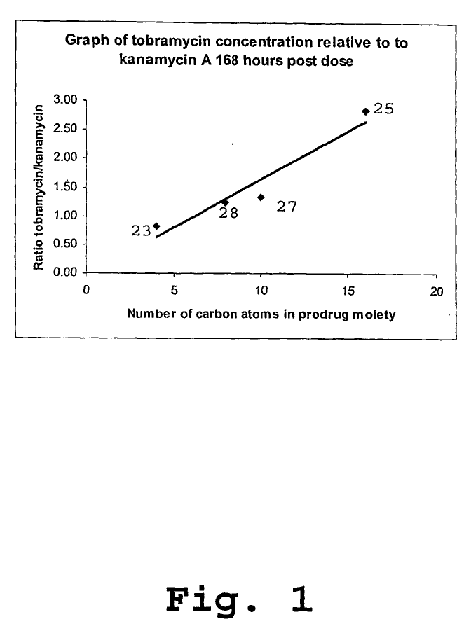

Since prodrugs were designed to increase aminoglycoside concentrations over extended periods of time, the data is expressed as the ratio of tobramycin to kanamycinA present 168 hours post dose. The data clearly indicates that for the homologous series of compounds 23, 25, 27 and 28, levels of tobramycin relative to kanamycinA are influenced by the nature of the prodrug component as shown in FIG. 1.

### REFERENCES

1) He G.; Massarella J.; Ward P. Clinical Pharmacokinetics of the Prodrug Oseltamivir and its Active Metabolite Ro 64-0802. Clin. Pharmacokinet., 1999, 37, 471-484.

2)

a) Hansch, C.; Bjorkroth, J. P.; Leo, A. Hydrophobicity and central nervous system agents: on the principle of minimal hydrophobicity in drug design. J. Pharm. Sci. 1987, 76, 663-687;

b) Driscoll, J. S.; Siddiqui, M. A.; Ford, H., Jr.; Kelley, J. A.; Roth, J. S.; Mitsuya, H.; Tanaka, M.; Marquez, V. E. Lipophilic, Acid-Stable, Adenosine Deaminase-Activated Anti-HIV Prodrugs for Central Nervous System Delivery. 3. 6-Amino Prodrugs of 2′-Fluoro-2′,3′-dideoxyinosine; J. Med. Chem., 1996, 39, 1619-1625.

3) Shechter, Y.; Tsubery, H.; Fridkin, M. N-[(2-Sulfo)-9-fluorenylmethoxycarbonyl]₃-gentamicin C₁ Is a Long-Acting Prodrug Derivative. J. Med. Chem., 2002; 45; 4264-4270.

4) Suzuki, H.; Kajimoto, Y.; Kumagai, H. Improvement of the Bitter Taste of Amino Acids through the Transpeptidation Reaction of Bacterial Glutamyltranspeptidase. J. Agric. Food Chem., 2002; 50, 313-318.

5) Connors, T. A., and Knox, R. J. Prodrugs in cancer chemotherapy. Sten Cells 1995 13, 501-11.

6) P Flecknell. Laboratory Animal Anesthesia, Second edition 1996, Academic Press, London, UK.

7) Niessen, W. M. A. Analysis of antibiotics by liquid chromatography-mass spectrometry. Journal of Chromatography, A 1998, 812(1+2), 53-75.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A prodrug of the general Formula (I), (II) or (III): 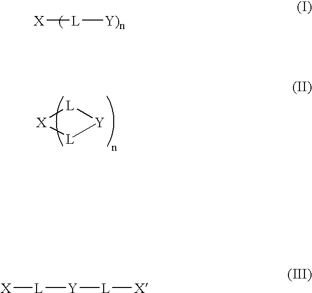
in which
X is a tobramycin moiety;
X′ is a pharmaceutically active moiety;
L is a linker group;
Y is a pharmacokinetic regulator; and
n is an integer of 1 or greater
or a pharmaceutically acceptable derivative or salt thereof.

2. A prodrug according to claim 1, wherein the pharmaceutically active moiety is selected from the group consisting of an aminoglycoside, nucleoside, rhinovirus capsid-binding compound, antisense oligonucleotide, peptide, an inhibitor of HIVRT, an inhibitor of influenza neuraminidase, amphotericin β, an azole and an aspartic proteinase.

3. A prodrug according to claim 2, wherein the aminoglycoside is selected from the group consisting of tobramycin, kanamycin A to C, amikacin, neomycin, streptomycin, neamine, paromomycin, lividomycin, 2230-C, ribostamycin, xyllostasin, butirosin, 4′-deoxybutyrosin, LL-BM408a, gentamycins and nebramycin.

4. A prodrug according to claim 3, wherein the aminoglycoside is selected from the group consisting of tobramycin, amikacin, neomycin and kanamycin.

5. A prodrug according to claim 3 or claim 4, wherein the aminoglycoside is tobramycin.

6. A prodrug according to claim 1, wherein the linker group is selected from the group consisting of esters, amides, ureas, thioureas, imines, acetals, ethers, phosphates, phosphate esters or diesters, thioesters, oximes and hydrazones.

7. A prodrug according to claim 6, wherein the linker group is selected from the group consisting of an ester, amide, oxime and phosphate.

8. A prodrug according claim 2, wherein the linker group is an ester.

9. A prodrug according to claim 1, wherein the pharmacokinetic regulator Y is a hydrophobic or hydrophilic moiety.

10. A prodrug according to claim 9, wherein the hydrophobic moiety is an optionally substituted straight chain, branched and/or cyclic saturated or unsaturated hydrocarbon.

11. A prodrug according to claim 10, wherein the hydrophobic moiety is an optionally substituted alkyl or optionally substituted alkenyl having 1 to 24 carbon atoms which is optionally interrupted with oxygen or nitrogen; an optionally substituted aryl; or an optionally substituted heterocyclyl.

12. A prodrug according to claim 11, wherein the optionally substituted alkyl or the optionally substituted alkenyl is an optionally substituted C₁₋₂₀ alkyl or optionally substituted C₂₋₂₀ alkenyl which is optionally interrupted with O, C═O, NH, optionally substituted aryl or optionally substituted heterocyclyl and optionally substituted with carboxyl, optionally substituted C₁₋₆ alkyl, amino or hydroxyl.

13. A prodrug according to claim 11, wherein the optionally substituted aryl is an optionally substituted phenyl or optionally substituted biphenyl.

14. A prodrug according to claim 11, wherein the optionally substituted heterocyclyl is a 5- or 6-membered nitrogen containing heterocyclic group.

15. A prodrug according to claim 14, wherein the heterocyclic group is selected from the group consisting of pyridyl, indolyl, indazolyl, 2,3-dihydro-1H-indolyl, furanyl, isoxazolyl, pyrazolyl and thiofuranyl.

16. A prodrug according to claim, wherein the optional substituents on the phenyl or heterocyclyl are selected from the group consisting of halo, C₁₋₄ alkyl, C₁₋₄ alkoxy, hydroxy and OCF₃.

17. A prodrug according to claim 9, wherein the hydrophilic moiety is selected from the group consisting of oligonucleotides up to 20 nucleotides in length, peptides up to 20 amino acids in length, peptide mimics, carbohydrates, oligosaccharides and derivatives thereof.

18. A method for the preparation of the prodrug of claim 1 comprising the steps of:
(a) optionally protecting the moieties X and/or X′ and/or the linker group which is attached to the optionally protected pharmacokinetic regulator Y;
(b) reacting the optionally protected moieties X and/or X′ and the optionally protected linker group L attached to the optionally protected pharmacokinetic regulator Y; and
(c) if necessary, removing the protecting groups of the moieties X and/or X′, the linker L and the pharmacokinetic regulator Y.

19. A pharmaceutical formulation comprising the prodrug of claim 1 or a pharmaceutically acceptable salt or derivative thereof, together with one or more pharmaceutically acceptable carriers.

20. A pharmaceutical formulation according to claim 19, which further comprises one or more other therapeutic and/or prophylactic ingredients.

21. A pharmaceutical formulation according to claim 20, wherein the other therapeutic and/or prophylactic ingredients is an antimicrobial or antiinfective agent.

22. A pharmaceutical formulation according to claim 21, wherein the antiinfective agent is an antibacterial agent.

23. A pharmaceutical formulation according to claim 22, wherein the antibacterial agent is effective to treat respiratory infections.

24. A pharmaceutical formulation according to claim 22, wherein the antibacterial agent is a combination selected from the group consisting of trimethoprim and sulfonamide; bacitracin and polymyxin B-neomycin; imipenem and fluoroquinolone; and beta-Iactam and aminoglycosides.

25. An inhaler which comprises a prodrug of claim 1.

26. An inhaler according to claim 25, wherein said inhaler is adapted for oral administration as a free-flow powder.

27. An inhaler according to claim 25, wherein said inhaler is a metered dose aerosol inhaler.

28. A method for the prevention and/or treatment of a microbial infection comprising the step of administration to a subject in need thereof of an effective amount of the prodrug of claim 1.

29. A method according to claim 28, wherein the microbial infection is a bacterial infection.

30. A method according to claim 29, wherein the infection is a Gram Negative or Gram Positive infection.

31. A method according to claim 30, wherein the bacterial infection is associated with the respiratory tract, urinary tract or GI tract or a systemic infection caused by enteric bacteria.

32. A method according to claim 28, wherein the administration is to the respiratory tract by inhalation, insufflation or intranasally or a combination thereof.

33-36. (canceled)

37. A method for the detection of a microbial infection which comprises the step of contacting the prodrug of claim 1 with a sample suspected of containing the microorganism.

38. A prodrug of general Formula (I), (II) or (III): 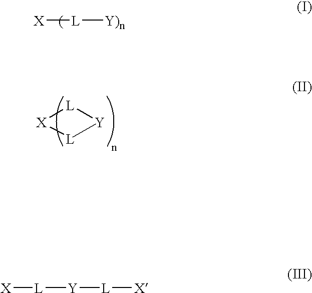
in which
X and X′ are either the same or different and selected from an aminoglycoside excluding tobramycin;
L is a linker group excluding amide and carbamate;
Y is a pharmacokinetic regulator; and
n is an integer of 1 or greater
or a pharmaceutically acceptable derivative or salt thereof.

39. A prodrug according to claim 38, wherein the aminoglycoside X is selected from the group consisting of kanamycin A to C, amikacin, neomycin, streptomycin, neamine, paromomycin, lividomycin, 2230-C, ribostamycin, xyllostasin, butirosin, 4′-deoxybutyrosin, LL-BM408a, gentamycins and nebramycin.

40. A prodrug according to claim 39, wherein the aminoglycoside is selected from the group consisting of amikacin, neomycin and kanamycin.

41. A prodrug according to claim 38, wherein the linker group is selected from the group consisting of esters, ureas, thioureas, imines, acetals, ethers, phosphates, phosphate esters or diesters, thioesters, oximes and hydrazones.

42. A prodrug according to claim 41, wherein the linker group is selected from the group consisting of an ester, oxime and phosphate.

43. A prodrug according to claim 41, wherein the linker group is an ester.

44. A prodrug according to claim 38, wherein the pharmacokinetic regulator is a hydrophobic or hydrophilic moiety.

45. A prodrug according to claim 44, wherein the hydrophobic moiety is an optionally substituted straight chain, branched and/or cyclic saturated or unsaturated hydrocarbon.

46. A prodrug according to claim 45, wherein the hydrophobic moiety is an optionally substituted alkyl optionally substituted alkenyl having 1 to 24 carbon atoms which is optionally interrupted with oxygen or nitrogen; an optionally substituted aryl; or an optionally substituted heterocyclyl.

47. A prodrug according to claim 46, wherein the optionally substituted alkyl or optionally substituted alkenyl is an optionally substituted C₁₋₂₀ alkyl or optionally substituted C₂₋₂₀ alkenyl which is optionally interrupted with O, C═O, NH, optionally sub$tituted aryl or optionally substituted heterocyclyl and optionally substituted with carboxyl, optionally substituted C₁₋₆ alkyl, amino or hydroxyl.

48. A prodrug according to claim 46, wherein the optionally substituted aryl is an optionally substituted phenyl or optionally substituted biphenyl.

49. A prodrug according to claim 46, wherein the optionally substituted heterocyclyl is a 5- or 6-membered nitrogen containing heterocyclic group.

50. A prodrug according to claim 49, wherein the heterocyclic group is selected from the group consisting of pyridyl, indolyl, indazolyl, 2,3-dihydro-1H-indolyl, furanyl, isoxazolyl, pyrazolyl and thiofuranyl.

51. A prodrug according to claim 48, wherein the optional substituents on the phenyl or heterocyclyl are selected from the group consisting of halo, C₁₋₄ alkyl, C₁₋₄ alkoxy, hydroxy and OCF₃.

52. A prodrug according to claim 44, wherein the hydrophilic moiety is selected from the group consisting of oligonucleotides up to 20 nucleotides in length, peptides up to 20 amino acids in length, peptide mimics carbohydrates, oligosaccharides and derivatives thereof.

53. A method for the preparation of the prodrug of claim 38, comprising the steps of:
(a) optionally protecting the moieties X and/or X′ and/or the linker group which is attached to the optionally protected pharmacokinetic regulator Y;
(b) reacting the optionally protected moieties X and/or X′ and the optionally protected linker group L attached to the optionally protected pharmacokinetic regulator Y; and
(c) if necessary, removing the protecting groups of the moieties X and/or X′ the linker L and the pharmacokinetic regulator Y.

54. A pharmaceutical formulation comprising the prodrug of claim 38 or a pharmaceutically acceptable salt or derivative thereof, together with one or more pharmaceutically acceptable carriers.

55. A pharmaceutical formulation according to claim 54, which further comprises one or more other therapeutic and/or prophylactic ingredients.

56. A pharmaceutical formulation according to claim 55, wherein the other therapeutic and/or prophylactic ingredients is an antimicrobial or antiinfective agent.

57. A pharmaceutical formulation according to claim 56, wherein the antiinfective agent is an antibacterial agent.

58. A pharmaceutical formulation according to claim 57, wherein the antibacterial agent is effective to treat respiratory infections.

59. A pharmaceutical formulation according to claim 57, wherein the antibacterial agent is a combination selected from the group consisting of trimethoprim and sulfonamide; bacitracin and polymyxin B-neomycin; imipenem and fluoroquinolone; and beta-lactam and aminoglycosides.

60. An inhaler which comprises a prodrug of claim 38.

61. An inhaler according to claim 60, wherein said inhaler is adapted for oral administration as a free-flow powder.

62. An inhaler according to claim 60, wherein said inhaler is a metered dose aerosol inhaler.

63. A method for the prevention and/or treatment of a microbial infection comprising the step of administration to a subject in need thereof of an effective amount of the prodrug of claim 38.

64. A method according to claim 63, wherein the microbial infection is a bacterial infection.

65. A method according to claim 64, wherein the bacterial infection is a Gram Negative or Gram Positive infection.

66. A method according to claim 65, wherein the bacterial infection is associated with the respiratory tract, urinary tract or GI tract or a systemic infection caused by enteric bacteria.

67. A method according to claim 63 wherein the administration is to the respiratory tract by inhalation, insufflation or intranasally or a combination thereof.

68-71. (canceled)

72. A method for the detection of a microbial infection which comprises the step of contacting the prodrug of claim 38 with a sample suspected of containing the microorganism.

73. A prodrug of the general Formula (I), (II) or (III): 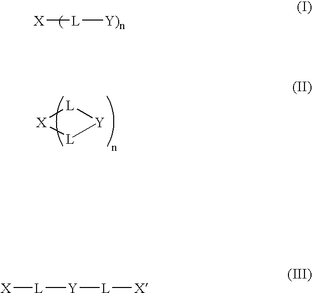
in which
X and X′ are either the same or different and selected from a nucleoside, rhinovirus capsid-binding compound, antisense oligonucleotide, peptide, an inhibitor of HIVRT, an inhibitor of influenza neuraminidase amphotericin β, an azole and an aspartic proteinase;
L is a linker group;
Y is a pharmacokinetic regulator; and
n is an integer of 1 or greater
or a pharmaceutically acceptable derivative or salt thereof.

74. A prodrug according to claim 73, wherein the linker group is selected from the group consisting of esters, amides, ureas, thioureas, imines, acetals, ethers, phosphates, phosphate esters or diesters, thioesters, oximes and hydra zones.

75. A prodrug according to claim 74, wherein the linker group is selected from the group consisting of an ester, amide, oxime and phosphate.

76. A prodrug according to claim 74, wherein the linker group is an ester.

77. A prodrug according to claim 73, wherein the pharmacokinetic regulator is a hydrophobic or hydrophilic moiety.

78. A prodrug according to claim 77, wherein the hydrophobic moiety is an optionally substituted straight chain, branched and/or cyclic saturated or unsaturated hydrocarbon.

79. A prodrug according to claim 78, wherein the hydrophobic moiety is an optionally substituted alkyl or optionally substituted alkenyl having 1 to 24 carbon atoms which is optionally interrupted with oxygen or nitrogen; an optionally substituted aryl; or an optionally substituted heterocyclyl.

80. A prodrug according to claim 79, wherein the optionally substituted alkyl or the optionally substituted alkenyl is an optionally substituted C₁₋₂₀ alkyl or optionally substituted C₂₋₂₀ alkenyl which is optionally interrupted with O, C═O, NH, optionally substituted aryl or optionally substituted heterocyclyl and optionally substituted with carboxyl, optionally substituted C₁₋₆ alkyl, amino or hydroxyl.

81. A prodrug according to claim 79, wherein the optionally substituted aryl is an optionally substituted phenyl or optionally substituted biphenyl.

82. A prodrug according to claim 79, wherein the optionally substituted heterocyclyl is a 5- or 6-membered nitrogen containing heterocyclic group.

83. A prodrug according to claim 82, wherein the heterocyclic group is selected from the group consisting of pyridyl, indolyl, indazolyl, 2,3-dihydro-1H-indolyl, furanyl, isoxazolyl pyrazolyl and thiofuranyl.

84. A prodrug according to claim 81, wherein the optional substituents on the phenyl or heterocyclyl are selected from the group consisting of halo, C₁₋₄ alkyl, C₁₋₄ alkoxy, hydroxy and OCF₃.

85. A prodrug according to claim 77, wherein the hydrophilic moiety is selected from the group consisting of oligonucleotides up to 20 nucleotides in length, peptides up to 20 amino acids in length, peptide mimics, carbohydrates, oligosaccharides and derivatives thereof.

86. A method for the preparation of the prodrug of claim 73 comprising the steps of:
(a) optionally protecting the moieties X and/or X′ and/or the linker group which is attached to the optionally protected pharmacokinetic regulator Y;
(b) reacting the optionally protected moieties X and/or X′ and the optionally protected linker group L attached to the optionally protected pharmacokinetic regulator Y; and
(c) if necessary, removing the protecting groups of the moieties X and/or X′, the linker L and the pharmacokinetic regulator Y.

87. A pharmaceutical formulation comprising the prodrug of claim 73 or a pharmaceutically acceptable salt or derivative thereof, together with one or more pharmaceutically acceptable carriers.

88. A pharmaceutical formulation according to claim 87, which further comprises one or more other therapeutic and/or prophylactic ingredients.

89. A pharmaceutical formulation according to claim 88, wherein the other therapeutic and/or prophylactic ingredients is an antimicrobial or antiinfective agent.

90. A pharmaceutical formulation according to claim 89, wherein the antiinfective agent is an antibacterial agent.

91. A pharmaceutical formulation according to claim 90, wherein the antibacterial agent is effective to treat respiratory infections.

92. A pharmaceutical formulation according to claim 90, wherein the antibacterial agent is a combination selected from the group consisting of trimethoprim and sulfonamide; bacitracin and polymyxin B-neomycin; imipenem and fluoroquinolone; and beta-lactam and aminoglycosides.

93. An inhaler which comprises a prodrug of claim 73.

94. An inhaler according to claim 93, wherein said inhaler is adapted for oral administration as a free-flow powder.

95. An inhaler according to claim 93, wherein said inhaler is a metered dose aerosol inhaler.

96. A method for the prevention and/or treatment of a microbial infection comprising the step of administration to a subject in need thereof of an effective amount of the prodrug of claim 73.

97. A method according to claim 96, wherein the microbial infection is a viral, fungal, parasitic, yeast or protozoal infection.

98. A method according to claim 97, wherein the viral infection is an orthomyxovirus or paramyxovirus infection.

99. A method according to claim 97, wherein the viral infection is an influenza A or B infection, parainfluenza, mumps or Newcastle disease.

100. A method according to claim 96, wherein the administration is to the respiratory tract by inhalation, insufflation or intranasally or a combination thereof.

101-104. (canceled)

105. A method for the detection of a microbial infection which comprises the step of contacting the prodrug of claim 73 with a sample suspected of containing the microorganism.
